(19) United States
(12) Patent Application Publication
(10) Pub. No.: US 2025/0248810 A1
(43) Pub. Date: Aug. 7, 2025

(54) COVERINGS FOR A PROSTHETIC HEART VALVE

(71) Applicant: EDWARDS LIFESCIENCES CORPORATION, Irvine, CA (US)

(72) Inventors: Roy Shitrit, Zichron Yaakov (IL); Brendan Michael Dalbow, Oceanside, CA (US); Nikolai Gurovich, Hadera (IL); Tamir S. Levi, Zikhron Yaakov (IL); Elena Sherman, Pardes Hana (IL); Maya Katz, Tel Aviv (IL)

(73) Assignee: EDWARDS LIFESCIENCES CORPORATION, Irvine, CA (US)

(21) Appl. No.: 19/190,479

(22) Filed: Apr. 25, 2025

Related U.S. Application Data
(63) Continuation of application No. PCT/US2023/079613, filed on Nov. 14, 2023.
(60) Provisional application No. 63/582,383, filed on Sep. 13, 2023.
(60) Provisional application No. 63/384,273, filed on Nov. 18, 2022.

(51) Int. Cl. A61F 2/24; A61F 2/91

(52) U.S. Cl. CPC A61F 2/2418; A61F 2/91; A61F 2210/0076; A61F 2220/0075

(57) ABSTRACT

Coverings and/or skirts for prosthetic heart valves are disclosed. As an example, a prosthetic heart valve comprises a frame comprising three axially extending commissure support portions, a plurality of rows of angled struts including a first row defining an inflow end, a second row downstream of the first row, and a third row downstream of the second row and defining an outflow end, where downstream ends of the struts of the second row are connected to each other at junctions, and the commissure support portions extend from respective junctions to the third row of struts. The valve further comprises leaflets forming three commissures secured to respective commissure support portions, and an outer skirt comprising an outflow edge forming three circumferentially spaced projections aligned with the commissure support portions and covering respective junctions, the outflow edge comprising straight sections extending between adjacent projections upstream of the junctions.

### CROSS-REFERENCE TO RELATED APPLICATIONS

This application is a continuation of PCT Application No. PCT/US2023/079613, filed Nov. 14, 2023, which claims the benefit of U.S. Provisional Patent Application Nos. 63/384,273, filed Nov. 18, 2022, and 63/582,383, filed Sep. 13, 2023, the entire contents of each of which are incorporated by reference herein.

### FIELD

The present disclosure relates to prosthetic heart valves, and in particular to coverings and/or skirts for prosthetic heart valves.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (e.g., stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (e.g., through a femoral artery and the aorta) until the prosthetic valve reaches the implantation site in the heart. The prosthetic valve is then expanded to its functional size, for example, by inflating a balloon on which the prosthetic valve is mounted, actuating a mechanical actuator that applies an expansion force to the prosthetic valve, or by deploying the prosthetic valve from a sheath of the delivery apparatus so that the prosthetic valve can self-expand to its functional size.

Most expandable, prosthetic heart valves comprise a cylindrical metal frame or stent and prosthetic leaflets mounted inside the frame. These valves can also include an outer covering or skirt disposed around an outer surface of the frame. The outer skirt can be configured to establish a seal with the native tissue and reduce perivalvular leakage when the prosthetic heart valve is placed at the implantation site (and thus may be referred to as sealing members). In some examples, these valves can additionally or alternatively include an inner skirt disposed around an inner surface of the frame.

### SUMMARY

Described herein are prosthetic heart valves, delivery apparatuses, and methods for implanting prosthetic heart valves. In particular, described herein are examples of outer skirts (or coverings) and/or inner skirts (or coverings) for prosthetic heart valves and methods of attaching such inner and/or outer skirts to a frame of a prosthetic heart valve. Prosthetic heart valves can include a frame and a leaflet assembly arranged on an inside of the frame. The prosthetic heart valve can include an outer skirt arranged around a circumference of the frame and on an outer surface of the frame. The outer skirt can include an outflow edge portion disposed upstream of commissure support portions of the frame where adjacent sides of adjacent leaflets are attached to the frame (forming commissures). In some examples, the outflow edge portion can form three circumferentially spaced projections aligned with the commissure support portions that are configured to cover a gap between a straight section of the outflow edge portion and the commissure support portions. In some examples, the prosthetic heart valve can include an inner skirt arranged around a circumference of the frame and on an inner surface of the frame, and the inner skirt can comprise an outflow edge portion with circumferentially spaced projections that are aligned with the commissure support portions of the frame (and cover gaps in the frame, as described above). In some examples, each commissure can include an attachment member and the attachment member can comprise an extension that extends axially upstream of a respective commissure support portion to cover the gap between the outflow edge portion of the outer skirt and the commissure support portion. Such outer coverings and/or inner coverings (the projections of the outer skirt, the projections of the inner skirt, or the extensions of the attachment members) can cover gaps in the frame that are not covered by the inner and/or outer skirts, thereby reducing perivalvular leakage through the frame when the prosthetic heart valve is implanted at a native tissue. In some examples, inner skirts can include features such as overlapping edge portions configured to be secured to a cusp edge portion of a leaflet of the prosthetic valve and/or folded portions along the outflow edge portion that, in some examples, can overlap an outflow edge of an outer skirt of the prosthetic valve. As such, the devices and methods disclosed herein can, among other things, overcome one or more of the deficiencies of typical prosthetic heart valves.

A prosthetic heart valve can comprise a frame and a valve structure coupled to the frame. In addition to these components, a prosthetic heart valve can further comprise one or more of the components disclosed herein.

In some examples, a prosthetic heart valve can comprise a sealing member configured to reduce paravalvular leakage, such as an outer skirt.

In some examples, a prosthetic heart valve can comprise an outer skirt comprising an inflow edge and an outflow edge, where the outflow edge forms three circumferentially spaced projections aligned with commissure support portions of the frame and covering gaps in the frame disposed between the commissure support portions and straight sections of the outflow edge, the straight sections extending between adjacent projections and located upstream of the commissure support portions.

In some examples, a prosthetic heart valve can comprise attachment members that form commissures with adjacent edges of the leaflets and attach to commissure support portions of the frame, where each attachment member comprises an extension that extends axially upstream of a respective commissure support portion to cover a gap between an outflow edge of the outer skirt and the commissure support portion.

In some examples, a prosthetic heart valve comprises a radially expandable and compressible frame comprising three axially extending commissure support portions and a plurality of rows of angled struts including a first row of angled struts defining an inflow end of the frame, a second row of angled struts downstream of the first row of angled struts, and a third row of angled struts downstream of the second row of angled struts and defining an outflow end of the frame. Downstream ends of the angled struts of the second row of angled struts are connected to each other at a plurality of junctions. The commissure support portions extend from respective junctions of the plurality of junctions of the second row of angled struts to the third row of angled struts. The prosthetic heart valve further comprises a plurality of leaflets forming three commissures secured to respective commissure support portions of the frame and an outer skirt disposed around an outer surface of the frame and comprising an inflow edge and an outflow edge. The outflow edge forms three circumferentially spaced projections aligned with the commissure support portions and covering respective junctions of the second row of angled struts, the outflow edge further comprising circumferentially extending sections extending between adjacent projections, where the circumferentially extending sections are located upstream of the junctions of the second row of angled struts.

In some examples, a prosthetic heart valve comprises a radially expandable and compressible frame comprising three commissure support portions and a plurality of rows of angled struts including a first row of angled struts defining an inflow end of the frame, a second row of angled struts downstream of the first row of angled struts, and a third row of angled struts downstream of the second row of angled struts and defining an outflow end of the frame. Downstream ends of the angled struts of the second row of angled struts are connected to each other at a plurality of junctions. The prosthetic heart valve further comprises a plurality of leaflets forming three commissures secured to respective commissure support portions of the frame, where upstream edges of the commissures are located adjacent respective junctions of the second row of struts. The prosthetic heart valve further comprises an outer skirt disposed around an outer surface of the frame and comprising an inflow edge and an outflow edge, where the outflow edge forms three circumferentially spaced projections aligned with the commissure support portions and covering respective junctions of the second row of angled struts. The outflow edge further comprises straight sections extending between adjacent projections, wherein the straight sections are located upstream of the junctions of the second row of angled struts.

In some examples, a prosthetic heart valve comprises a radially expandable and compressible frame comprising three commissure support portions and a plurality of rows of angled struts including a first row of angled struts defining an inflow end of the frame, a second row of angled struts downstream of the first row of angled struts, and a third row of angled struts downstream of the second row of angled struts and defining an outflow end of the frame. Downstream ends of the angled struts of the second row of angled struts are connected to each other at a plurality of junctions. The prosthetic heart valve further comprises a plurality of leaflets forming three commissures secured to respective commissure support portions of the frame, where upstream edges of the commissures are located adjacent respective junctions of the second row of angled struts. The prosthetic heart valve further comprises an outer skirt disposed around an outer surface of the frame and comprising an inflow edge and an outflow edge, where the outflow edge is located upstream of the junctions of the second row of angled struts. The prosthetic heart valve further comprises a plurality of commissure extension members located at respective commissure support portions on an outer surface of the frame, each commissure extension member comprising an extension that extends axially upstream of the junctions to cover a gap in the frame that is formed between the respective junction and the outflow edge of the outer skirt.

In some examples, a prosthetic heart valve comprises a frame comprising a plurality of interconnected struts and an inner skirt disposed around an inner surface of the frame. The inner skirt comprises an inflow edge portion and an outflow edge portion; and opposing first and second edge portions that each extend between the inflow edge portion and the outflow edge portion and are non-perpendicular to the inflow edge portion, wherein the first and second edge portions overlap one another to form an annular configuration of the inner skirt inside the frame. The prosthetic heart valve further comprises a plurality of leaflets arranged inside the frame, wherein a cusp edge portion of a first leaflet of the plurality of leaflets is arranged along the overlapping first and second edge portions; and a plurality of stitches securing the overlapping first and second edge portions and the cusp edge portion of the first leaflet to one another. In some examples, the plurality of stitches are in-and-out stitches.

In some examples, a prosthetic heart valve comprises a frame comprising a plurality of interconnected struts forming multiple rows of open cells between an outflow end and an inflow end of the frame; an outer skirt disposed around an outer surface of the frame and comprising an inflow edge and an outflow edge; and an inner skirt disposed around an inner surface of the frame. The inner skirt comprises an inflow edge portion and an outflow edge portion, the outflow edge portion extending through cells of the multiple rows of open cells of the frame to an exterior of the frame and folded over the outflow edge of the outer skirt.

In some examples, a prosthetic heart valve comprises a radially expandable and compressible frame comprising a plurality of interconnected struts, the frame having an inflow end and an outflow end; a plurality of leaflets arranged inside the frame; and an inner skirt disposed around an inner surface of the frame. The inner skirt comprises an inflow edge portion and an outflow edge portion, where the outflow edge portion is folded over itself such that it forms a folded edge, and a free edge of the outflow edge portion is arranged against a surface of the inner skirt that faces away from the leaflets.

In some examples, a prosthetic heart valve comprises one or more of the components recited in Examples 1-61, and 69-92 below.

A method for assembling a prosthetic heart valve can comprise arranging an inner skirt for the prosthetic heart valve into an annular configuration such that opposing first and second edge portions of the inner skirt overlap one another, arranging a cusp edge portion of a first leaflet of a plurality of leaflets of the prosthetic heart valve along the overlapping first and second edge portions, and securing the overlapping first and second edge portions and the cusp edge portion of the first leaflet to one another.

In some examples, the first and second edge portions extend between an inflow edge portion and an outflow edge portion of the inner skirt at an angle of less than 90 degrees.

In some examples, the overlapping first and second edge portions and the cusp edge portion of the first leaflet are secured to one another with a plurality of in-and-out stitches.

In some examples, the plurality of in-and-out stitches extend through aligned apertures extending through the first and second edge portions and the cusp edge portion of the first leaflet.

In some examples, a method for assembling a prosthetic heart valve comprises arranging an inner skirt for the prosthetic heart valve into an annular configuration such that opposing first and second edge portions of the inner skirt overlap one another, the first and second edge portions extending between an inflow edge portion and an outflow edge portion of the inner skirt at an angle of less than 90 degrees; arranging a cusp edge portion of a first leaflet of a plurality of leaflets of the prosthetic heart valve along the overlapping first and second edge portions; and securing the overlapping first and second edge portions and the cusp edge portion of the first leaflet to one another with a plurality of in-and-out stitches.

In some examples, a method comprises one or more of the components recited in Examples 62-68 below.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1 is a side view of a prosthetic heart valve, according to an example.

FIG. 2 is a side view of a frame of the prosthetic heart valve of FIG. 1.

FIG. 3 is a side view of a portion of the frame of FIG. 2, showing the portion of the frame in a straightened (non-annular) state.

FIG. 4 is a side view of an exemplary delivery apparatus configured to deliver and implant a radially expandable prosthetic heart valve at an implantation site.

FIG. 5 is a perspective view of a prosthetic heart valve, according to an example.

FIG. 6 is a side view of a portion of a frame for a prosthetic heart valve with an outer skirt disposed around an outer surface of the frame, the outer skirt comprising an outflow edge forming circumferentially spaced projections that extend from straight sections of the outflow edge toward a commissure support portion of the frame.

FIG. 7 is a side view of a portion of a frame for a prosthetic heart valve with an outer skirt disposed around an outer surface of the frame, the outer skirt comprising an outflow edge forming circumferentially spaced projections that extend from straight sections of the outflow edge toward a commissure support portion of the frame, where each projection is formed by stretching the outflow edge and securing the projection to the respective commissure support portion.

FIG. 8 is flattened view of an exemplary commissure attachment member comprising a main body and an extension extending away from the main body, the main body configured form a commissure and be secured to a commissure support portion of a frame of a prosthetic heart valve.

FIG. 9 is a side view of the attachment member of FIG. 8 in a folded configuration around axially extending window struts of a frame of a prosthetic heart valve.

FIG. 10 is a side view of a portion of a frame for a prosthetic heart valve with the attachment member of FIG. 8 folded around and coupled to axially extending window struts of the frame and the extension of the attachment member secured to an outflow edge of an outer skirt of the prosthetic heart valve.

FIG. 11 is a side view of an exemplary commissure attachment member in a folded configuration around axially extending window struts of a frame of a prosthetic heart valve, the attachment member split into two pieces configured to couple to different leaflets of two adjacent leaflets of the prosthetic heart valve.

FIG. 12 is a side view of a portion of a frame for a prosthetic heart valve with an inner skirt disposed around an inner surface of the frame, the inner skirt comprising an outflow edge portion forming circumferentially spaced projections that extend from straight sections of the outflow edge portion toward a commissure support portion of the frame.

FIG. 13 is a side view of an inner skirt for a prosthetic heart valve, according to an example, the inner skirt shown in a flattened configuration.

FIG. 14 is a side view of a portion of the inner skirt of FIG. 13 depicting opposing angled edge portions of the inner skirt overlapping one another and a cusp edge portion of a leaflet for a prosthetic valve, and stitches securing together the overlapping edge portions of the inner skirt and cusp edge portion of the leaflet.

FIG. 15 is a cross-sectional view of the inner skirt of FIG. 13 arranged against an inner surface of a frame of a prosthetic heart valve and in-and-out stitches extending through the two overlapping edge portions of the inner skirt and the cusp edge portion of the leaflet.

FIG. 16 is a side view of a prosthetic valve including a frame, the inner skirt of FIG. 13 disposed around an inner surface of the frame, and an outer skirt disposed around an outer surface of the frame, the inner skirt comprising projections that extend from straight sections of the outflow edge portion of the inner skirt toward commissure support portions of the frame.

FIG. 17 is a side view of a portion of the inner skirt of FIG. 13 with one of its straight sections folded over to form a folded portion at the outflow edge portion of the inner skirt.

FIG. 18 is a side view of another portion of the inner skirt of FIG. 13 with straight sections of the outflow edge portion folded over the inner skirt and a leaflet secured to the inner skirt along its cusp edge portion.

FIG. 19 is a side view of a portion of a frame of a prosthetic valve with an outer skirt disposed around an outer surface of the frame, and an inner skirt disposed around the inner surface with inflow and outflow edge portions folded over corresponding inflow and outflow edges of the outer skirt.

FIG. 20 is a side view of an inner skirt for a prosthetic heart valve, according to an example, the inner skirt shown in a flattened configuration and comprising slits along the straight sections of the outflow edge portion.

FIG. 21 is a cross-sectional view of a frame for a prosthetic valve, an outer skirt disposed against an outer surface of the frame, and an inner skirt disposed against an inner surface of the frame, where inflow and outflow edge portions of the inner skirt extend around and through, respectively, the frame and overlap corresponding inflow and outflow edges of the outer skirt.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (e.g., out of the patient's body), while distal motion of the device is motion of the device away from the user and toward the implantation site (e.g., into the patient's body). The terms “longitudinal” and “axial” refer to an axis extending in the proximal and distal directions, unless otherwise expressly defined.

As used herein, “e.g.” means “for example,” and “i.e.” means “that is.”

As used herein, “upstream” and “downstream” refers to a position, direction, or portion of a prosthetic valve that is relative to an outflow end and an inflow end of the prosthetic valve and/or a frame of the prosthetic valve and a direction of blood flow through the prosthetic valve (from the inflow end to the outflow end). For example, the outflow end of the prosthetic valve and/or frame is the downstream end of the prosthetic valve and/or frame and the inflow end of the prosthetic valve and/or frame is the upstream end of the prosthetic valve and/or frame.

### Overview of the Disclosed Technology

As introduced above, prosthetic heart valves can include a frame comprising interconnected struts, leaflets mounted inside the frame, and an outer skirt disposed around an outer surface of the frame. In some examples, the prosthetic heart valve can include an inner skirt disposed around an inner surface of the frame. The frame can comprise a plurality of rows of angled struts and a plurality of commissure support portions. The leaflets form commissures that are secured to respective commissure support portions of the frame. Each commissure support portion can extend between or be formed at least in part by angled struts of a first row of angled struts defining an outflow end of the frame and angled struts of a second row of angled struts that are disposed upstream of the first row of angled struts.

In some examples, an outflow edge of the outer skirt and an outflow edge of the inner skirt (if included) are disposed upstream of junctions that are formed between the angled struts of the second row of angled struts and that connect to the commissure support portions of the frame. As a result, a gap is formed between the outflow edge of the outer skirt (and/or inner skirt) and the commissure support portions of the frame through which blood can flow following implantation of the prosthetic heart valve (referred to as “perivalvular leakage” or “PVL”). While this configuration may reduce a crimp profile of the prosthetic heart valve (when in a radially compressed configuration mounted on a delivery apparatus), perivalvular leakage can occur through the gaps, thereby reducing an efficiency of the prosthetic heart valve.

Described herein are outer skirts with an outflow edge that forms three circumferentially spaced projections, each disposed between adjacent straight sections of the outer skirt. The projections are aligned with the commissure support portions and cover the junctions between the second row of angled struts that connect to the commissure support portions. As a result, a gap formed between the straight portions of the outflow edge of the outer skirt and the commissure support portions can be covered by the projections, thereby reducing perivalvular leakage.

Described also herein are attachment members that form commissures with adjacent edges of the leaflets and attach to the commissure support portions of the frame, where each attachment member comprises an extension that extends axially upstream of a respective commissure support portion to cover a gap between the outflow edge of the outer skirt and the commissure support portion. In this way, perivalvular leakage is reduced.

Described also herein are inner skirts with an outflow edge that forms three circumferentially spaced projections, each disposed between adjacent straight sections of the outer skirt. The projections are aligned with the commissure support portions and extend to the junctions between the second row of angled struts that connect to the commissure support portions. As a result, a gap formed between the straight portions of the outflow edge of the inner skirt and the commissure support portions can be covered by the projections, thereby reducing perivalvular leakage.

In some examples, the inner skirt can include additional features, such as angled end portions that overlap one another in a circumferential arrangement of the inner skirt. During assembly of the prosthetic heart valve, the overlapping angled end portions can also overlap a cusp edge portion of a leaflet and the resulting three overlapping layers can be secured together with the same suture (or sutures).

In some examples, an inflow edge portion and/or an outflow edge portion can be folded over the corresponding edges of the outer skirt, on an outside of the frame. As a result, outwardly protruding fibers of the outer skirt are prevented from extending inward, towards the leaflets.

Prosthetic valves disclosed herein can be radially compressible and expandable between a radially compressed state and a radially expanded state. Thus, the prosthetic valves can be crimped on or retained by an implant delivery apparatus in the radially compressed state while being advanced through a patient's vasculature on the delivery apparatus. The prosthetic valve can be expanded to the radially expanded state once the prosthetic valve reaches the implantation site. It is understood that the prosthetic valves disclosed herein may be used with a variety of implant delivery apparatuses and can be implanted via various delivery procedures, examples of which will be discussed in more detail later.

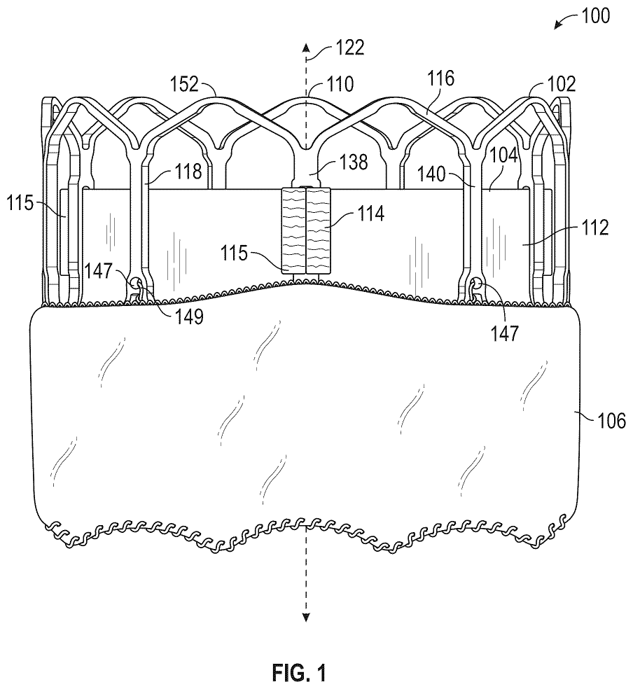

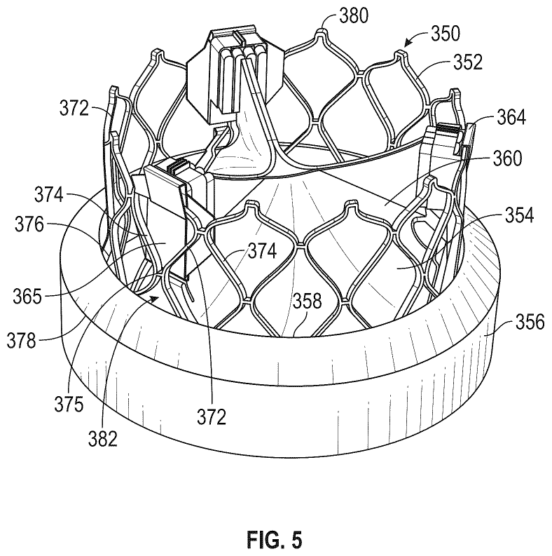

FIG. 1 illustrates an exemplary prosthetic heart valve comprising a frame, leaflets secured on an inside of the frame, and an outer skirt disposed around an outer surface of the frame. In some examples, the frame can comprise a plurality of interconnected and angled struts, including struts that form commissure windows (commissure support portions of the frame) that are configured to receive commissures of the leaflets, as shown in FIGS. 1-3. In some examples, a frame for a prosthetic heart valve can comprise commissure support portions configured to receive commissures of the leaflets, each commissure support portion formed by angled struts forming an open cell of the frame, as shown in FIG. 5. The prosthetic heart valves can be advanced through a patient's vasculature, such as to a native heart valve, by a delivery apparatus, such as the exemplary delivery apparatus shown in FIG. 4.

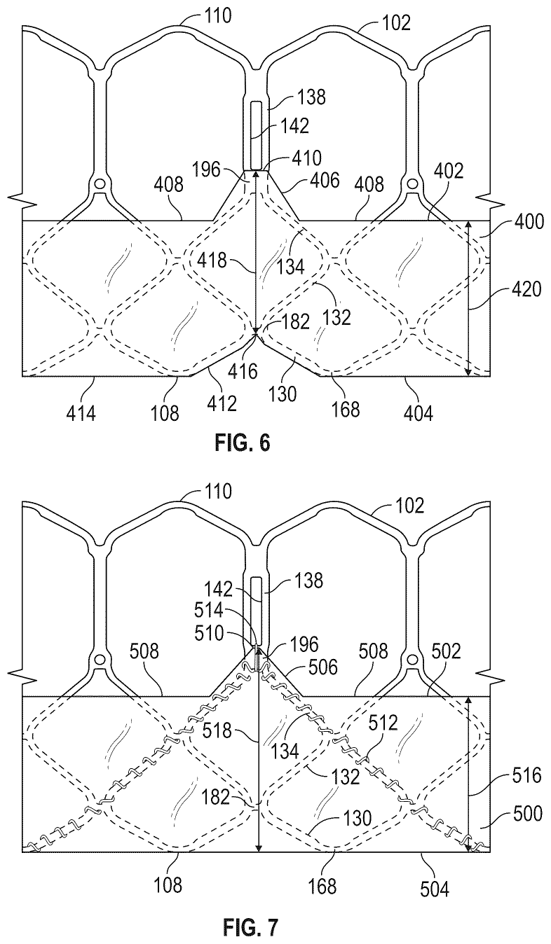

In some examples, an outer skirt for a prosthetic valve can comprise an outflow edge that forms three circumferentially spaced projections, each disposed between adjacent circumferentially extending sections of the outer skirt, as shown in FIGS. 6 and 7. In some examples, these projections are pre-formed in the outer skirt (FIG. 6). In some examples, these projections are formed by stretching the outflow edge and securing the stretched portions to the commissure support portions to form the projections. In both cases, the projections can cover gaps disposed in the frame between the commissure support portions and the circumferentially extending sections of the outflow edge of the outer skirt. In this way paravalvular, leakage can be reduced.

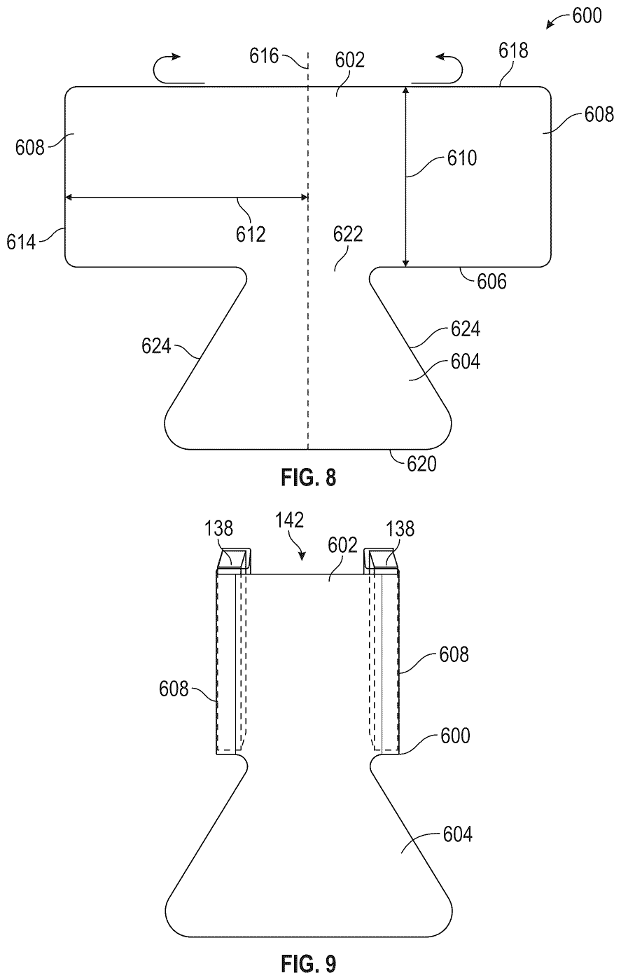

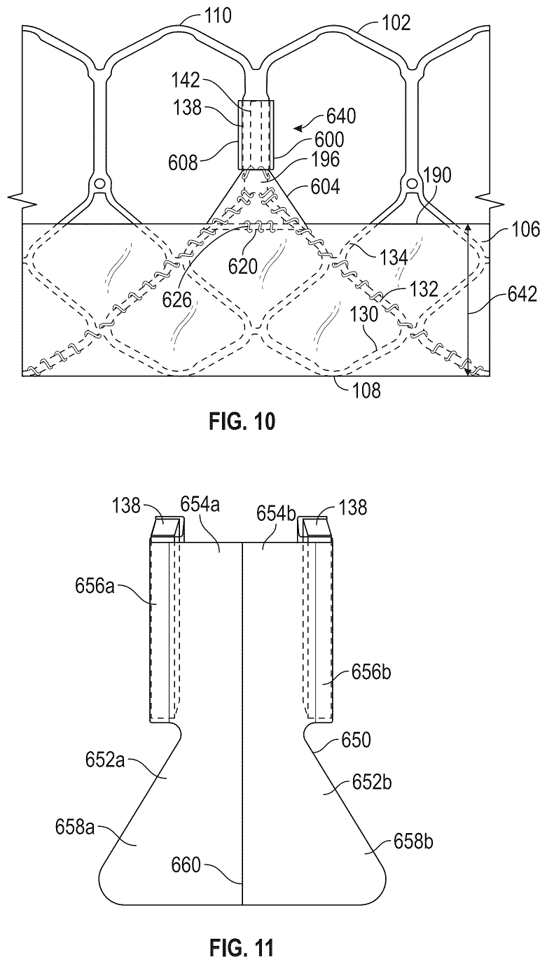

In some examples, a commissure attachment member can comprise a main body portion and an extension extending away from the main body portion, as shown in FIG. 8. The main body portion can comprise laterally extending flaps that are configured to fold around and attach to axially extending window struts and leaflets of a prosthetic valve (FIGS. 9 and 10). In some examples, the attachment member can be formed as two separate pieces that are configured to pair separately with adjacent edges of two adjacent leaflets (FIG. 11). The extension of the attachment member can attach to the outflow edge of the outer skirt of the prosthetic valve, thereby covering gaps in the frame and reducing perivalvular leakage (FIG. 10).

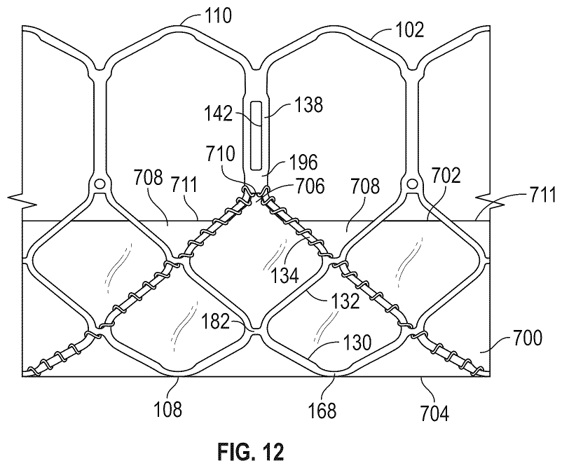

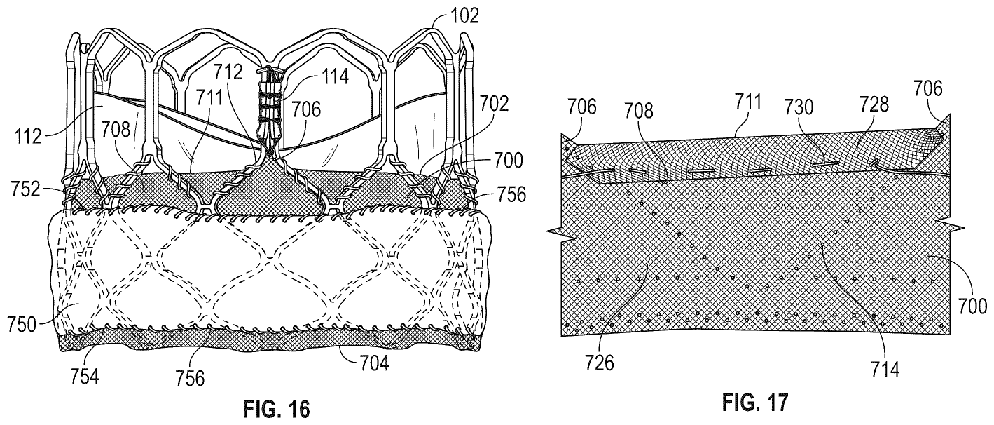

In some examples, an inner skirt for a prosthetic valve can comprise an outflow edge that forms three circumferentially spaced projections, each disposed between adjacent circumferentially extending sections of the outer skirt, as shown in FIGS. 12-16. The projections can cover gaps disposed in the frame between the commissure support portions and the circumferentially extending sections of the outflow edge of the inner skirt, as shown in FIGS. 12 and 16. In this way, paravalvular leakage can be reduced.

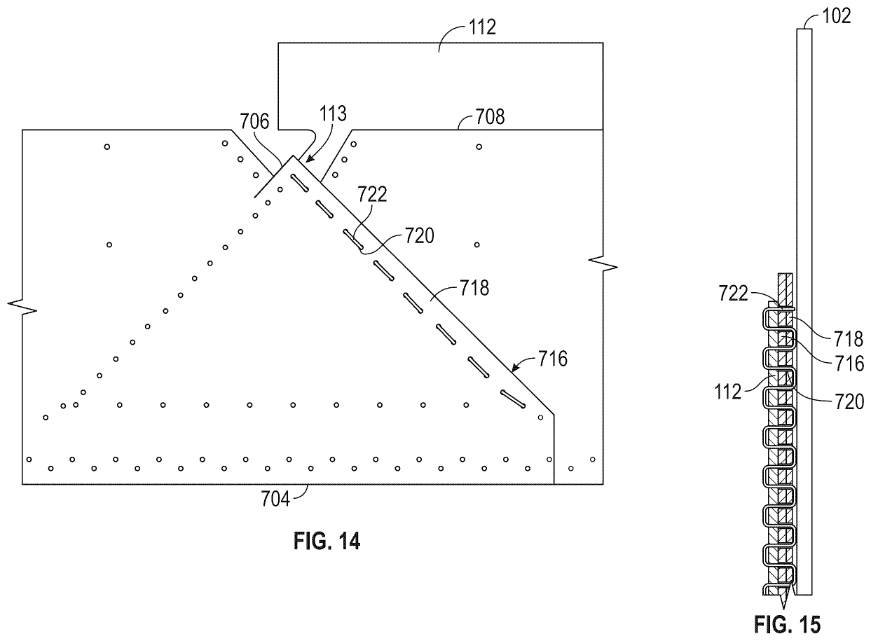

In some examples, an inner skirt can comprise angled edge portions that are configured to overlap one another when the inner skirt is arranged in an annular configuration inside a frame of a prosthetic valve. For example, as shown in FIGS. 14 and 15, the overlapping edge portions of the inner skirt can be secured to a cusp edge portion of a leaflet of the prosthetic heart valve, thereby simplifying the valve assembly process.

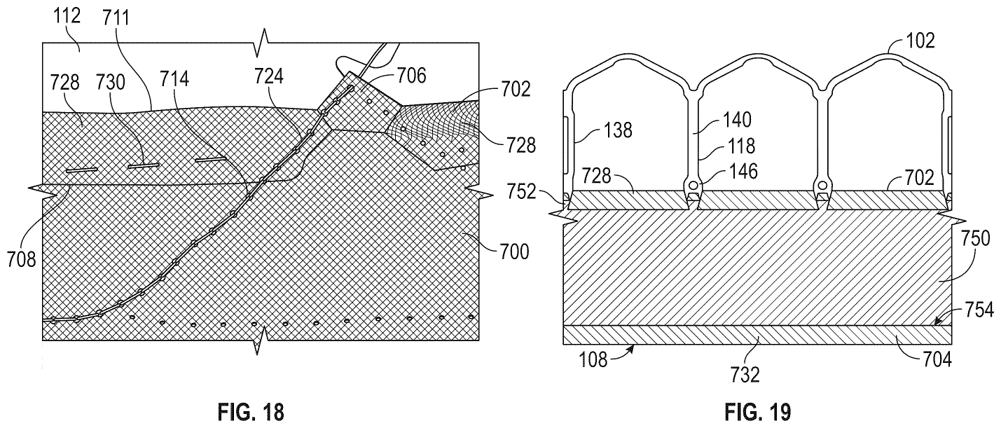

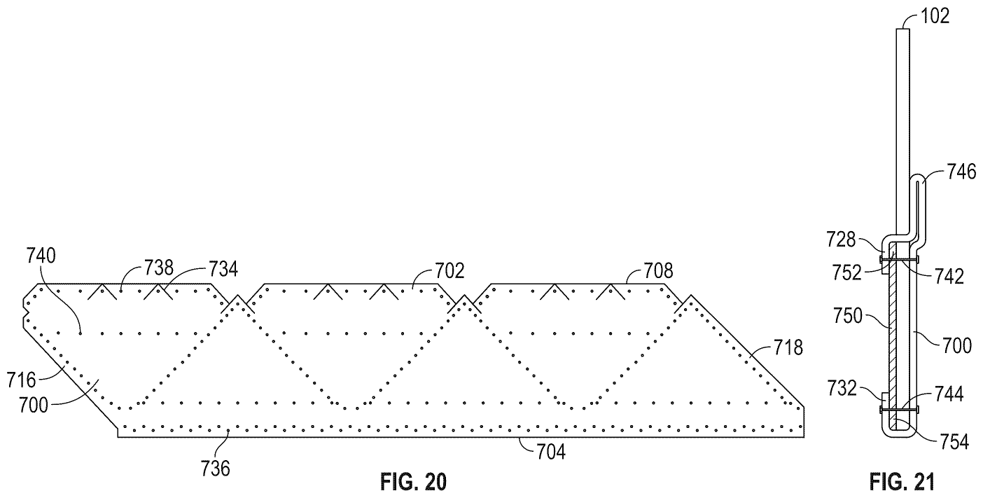

In some examples, an outflow edge portion of the inner skirt can be folded over, as shown in FIGS. 17 and 18, such that a free edge of the outflow edge portion is disposed against an outer surface of the inner skirt (the surface that faces the frame). In some examples, as shown in FIGS. 19 and 21, the folded portion or portions of the outer skirt can be folded over and cover an outflow edge of an outer skirt disposed around an outer surface of the frame of the prosthetic valve. In some examples, as shown in FIG. 20, the outflow edge portion of the inner skirt can comprise slits that allow multiple folded portions to be formed and extended through open cells in the frame, to the exterior of the frame. In some examples, an inflow edge portion of the inner skirt can be folded around the frame and over an inflow edge of the outer skirt, as shown in FIGS. 19 and 21.

### Examples of the Disclosed Technology

FIG. 1 shows a prosthetic heart valve 100 (prosthetic valve), according to an example. Any of the prosthetic valves disclosed herein are adapted to be implanted in the native aortic annulus, although in other examples they can be adapted to be implanted in the other native annuluses of the heart (the pulmonary, mitral, and tricuspid valves). The disclosed prosthetic valves also can be implanted within vessels communicating with the heart, including a pulmonary artery (for replacing the function of a diseased pulmonary valve, or the superior vena cava or the inferior vena cava (for replacing the function of a diseased tricuspid valve) or various other veins, arteries and vessels of a patient. The disclosed prosthetic valves also can be implanted within a previously implanted prosthetic valve (which can be a prosthetic surgical valve or a prosthetic transcatheter heart valve) in a valve-in-valve procedure.

In some examples, the disclosed prosthetic valves can be implanted within a docking or anchoring device that is implanted within a native heart valve or a vessel. For example, in one example, the disclosed prosthetic valves can be implanted within a docking device implanted within the pulmonary artery for replacing the function of a diseased pulmonary valve, such as disclosed in U.S. Publication No. 2017/0231756, which is incorporated by reference herein. In another example, the disclosed prosthetic valves can be implanted within a docking device implanted within or at the native mitral valve, such as disclosed in PCT Publication No. WO2020/247907, which is incorporated herein by reference. In another example, the disclosed prosthetic valves can be implanted within a docking device implanted within the superior or inferior vena cava for replacing the function of a diseased tricuspid valve, such as disclosed in U.S. Publication No. 2019/0000615, which is incorporated herein by reference.

The prosthetic heart valve 100 can include a stent or frame 102, a valvular structure 104, and a perivalvular outer sealing member or outer skirt 106. The prosthetic heart valve 100 (and the frame 102) can have an inflow end 108 and an outflow end 110. The valvular structure 104 can be disposed on an interior of the frame 102 while the outer skirt 106 is disposed around an outer surface of the frame 102.

The valvular structure 104 can comprise a plurality of leaflets 112 (e.g., three leaflets, as shown in FIG. 1), collectively forming a leaflet structure, which can be arranged to collapse in a tricuspid arrangement. The leaflets 112 can be secured to one another at their adjacent sides (e.g., commissure tabs) to form commissures 114 of the valvular structure 104. For example, each leaflet 112 can comprise opposing commissure tabs disposed on opposite sides of the leaflet 112 and a cusp edge portion extending between the opposing commissure tabs. The cusp edge portion of the leaflets 112 can have an undulating, curved scalloped shape, and can be secured directly to the frame 102 (e.g., by sutures). However, in alternate examples, the cusp edge portion of the leaflets 112 can be secured to an inner skirt which is then secured to the frame 102. In some examples, the leaflets 112 can be formed of pericardial tissue (e.g., bovine pericardial tissue), biocompatible synthetic materials, or various other suitable natural or synthetic materials as known in the art and described in U.S. Pat. No. 6,730,118, which is incorporated by reference herein.

In some examples, the outer skirt 106 can be an annular skirt. In some instances, the outer skirt 106 can comprise one or more skirt portions that are connected together and/or individually connected to the frame 102. The outer skirt 106 can comprise a fabric or polymeric material, such as ePTFE, PTFE, PET, TPU, UHMWPE, PEEK, PE, etc. In some instances, instead of having a relatively straight upper edge portion, as shown in FIG. 1, the outer skirt 106 can have an undulating upper edge portion that extends along and is secured to the angled struts 134. Examples of such outer skirts, as well as various other outer skirts, that can be used with the frame 102 can be found in U.S. provisional patent application No. 63/366,599 filed Jun. 17, 2022, which is incorporated by reference herein.

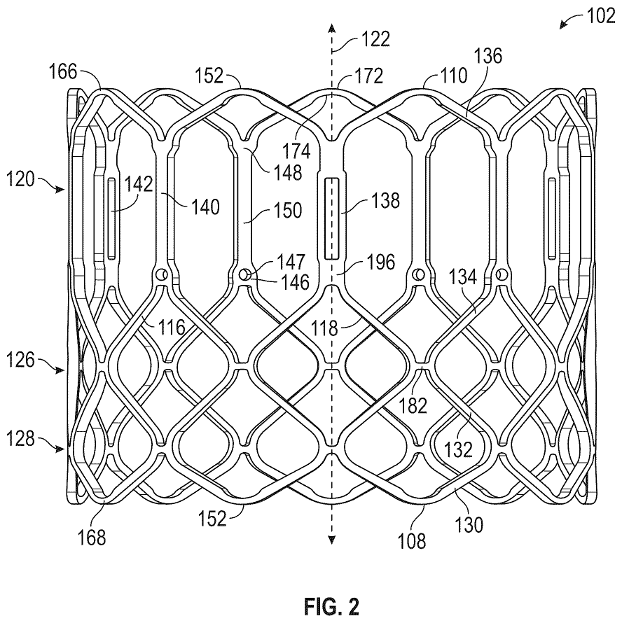

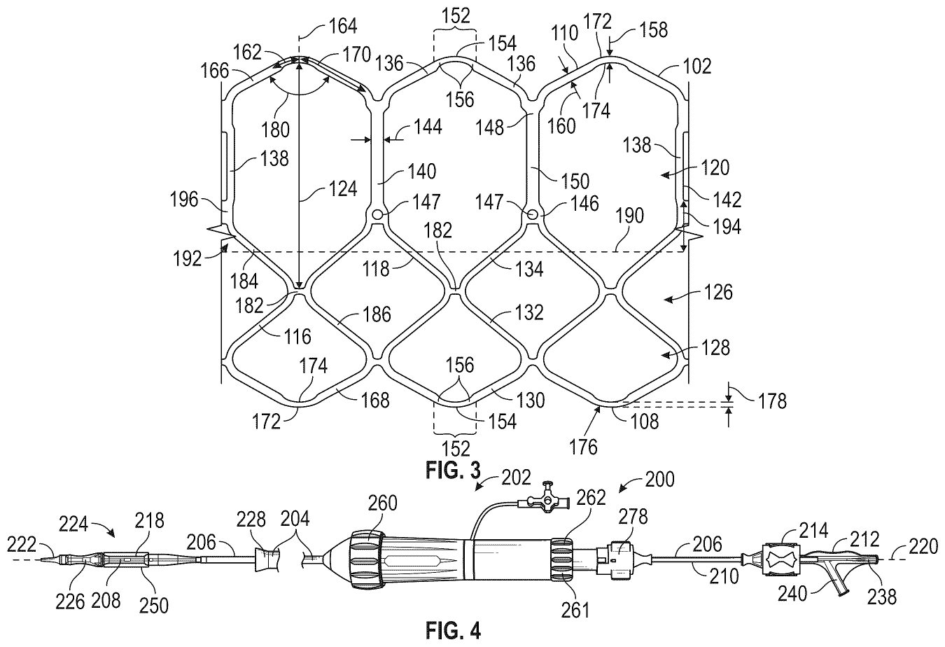

The frame 102 can be radially compressible and expandable between a radially compressed (or collapsed) configuration and a radially expanded configuration (the expanded configuration is shown in FIG. 1). The frame 102 is shown alone in FIG. 2 and a portion of the frame 102 in a straightened (non-annular) configuration is shown in FIG. 3.

The frame 102 can be made of any of various suitable plastically-expandable materials (e.g., stainless steel, etc.) or self-expanding materials (e.g., Nitinol). When constructed of a plastically-expandable material, the frame 102 (and thus the valve 100) can be crimped to a radially compressed state on a delivery catheter and then expanded inside a patient by an inflatable balloon or equivalent expansion mechanism. When constructed of a self-expandable material, the frame 102 (and thus the valve 100) can be crimped to a radially compressed state and restrained in the compressed state by insertion into a sheath or equivalent mechanism of a delivery catheter. Once inside the body, the valve can be advanced from the delivery sheath, which allows the valve to expand to its functional size.

Suitable plastically-expandable materials that can be used to form the frame 102 include, without limitation, metal alloys, polymers, or combinations thereof. Example metal alloys can comprise one or more of the following: nickel, cobalt, chromium, molybdenum, titanium, or other biocompatible metal. In some examples, the frame 102 can comprise stainless steel. In some examples, the frame 102 can comprise cobalt-chromium. In some examples, the frame 102 can comprise nickel-cobalt-chromium. In some examples, the frame 102 comprises a nickel-cobalt-chromium-molybdenum alloy, such as MP35N™ (tradename of SPS Technologies), which is equivalent to UNS R 30035 (covered by ASTM F562-02). MP35N™/UNS R30035 comprises 35% nickel, 35% cobalt, 20% chromium, and 10% molybdenum, by weight.

As shown in FIGS. 2 and 3, the frame 102 can comprise a plurality of interconnected struts 116 which form multiple rows of open cells 118 between the outflow end 110 and the inflow end 108 of the frame 102. In some examples, as shown in FIGS. 2 and 3, the frame 102 can comprise three rows of cells 118 with a first (upper in the orientation shown in FIGS. 2 and 3) row of cells 120 disposed at the outflow end 110. The first row of cells 120 comprises cells 118 that are elongated in an axial direction (relative to a central longitudinal axis 122 of the frame 102), as compared to cells 118 in the remaining rows of cells. For example, the cells 118 of the first row of cells 120 can have a longer axial length 124 (FIG. 3) than cells 118 in the remaining rows of cells, which can include a second row of cells 126 and a third row of cells 128, the third row of cells 128 disposed at the inflow end 108 and the second row of cells 126 disposed between the first row of cells 120 and the third row of cells 128.

In some examples, as shown in FIG. 2, each row of cells comprises nine cells 118. Thus, in such examples, the frame 102 can be referred to as a nine-cell frame.

In alternate examples, the frame 102 can comprise more than three rows of cells (e.g., four or five) and/or more or less than nine cells per row. In some examples, the cells 118 in the first row of cells 120 may not be elongated compared to cells 118 in the remaining rows of cells of the frame 102 (the second row of cells 126 and the third row of cells 128).

The interconnected struts 116 can include a plurality of angled struts 130, 132, 134, and 136 arranged in a plurality of rows of circumferentially extending rows of angled struts, with the rows being arrayed along the length of the frame 102 between the outflow end 110 and the inflow end 108. For example, the frame 102 can comprise a first row of angled struts 130 arranged end-to-end and extending circumferentially at the inflow end 108 of the frame; a second row of circumferentially extending, angled struts 132; a third row of circumferentially extending, angled struts 134; and a fourth row of circumferentially extending, angled struts 136 at the outflow end 110 of the frame 102. The fourth row of angled struts 136 can be connected to the third row of angled struts 134 by a plurality of axially extending window struts 138 (or window strut portions) and a plurality of axial (or axially extending) struts 140. The axially extending window struts 138 (which can also be referred to as axial struts that include a commissure window) define commissure windows (e.g., open windows) 142 that are spaced apart from one another around the frame 102, in a circumferential direction, and which are adapted to receive a pair of commissure tabs of a pair of adjacent leaflets 112 arranged into a commissure (e.g., commissure 114 shown in FIG. 1). In some examples, the commissure windows 142 and/or the axially extending window struts 138 defining the commissure windows 142 can be referred to herein as commissure features or commissure supports, each commissure feature or support configured to receive and/or be secured to a pair of commissure tabs of a pair of adjacent leaflets.

One or more (for example, two, as shown in FIGS. 2 and 3) axial struts 140 can be positioned between, in the circumferential direction, two commissure windows 142 formed by the window struts 138. Since the frame 102 can include fewer cells per row (e.g., nine) and fewer axial struts 140 between each commissure window 142, as compared to some more traditional prosthetic heart valves, each cell 118 can have an increased width (in the circumferential direction), thereby providing a larger opening for blood flow and/or coronary access.

Each axial strut 140 and each window strut 138 extends from a location defined by the convergence of the lower ends (e.g., ends arranged inward of and farthest away from the outflow end 110) of two angled struts 136 (which can also be referred to as an upper strut junction or upper elongated strut junction) to another location defined by the convergence of the upper ends (e.g., ends arranged closer to the outflow end 110) of two angled struts 134 (which can also be referred to as a lower strut junction or lower elongate strut junction). Each axial strut 140 and each window strut 138 forms an axial side of two adjacent cells of the first row of cells 120.

In some examples, as shown in FIG. 3, each axial strut 140 can have a width 144 (FIG. 3) that is larger than a width of the angled struts 130, 132, 134, and 136. As used herein, a “width” of a strut is measured between opposing locations on opposing surfaces of a strut that extend between the radially facing inner and outer surfaces of the strut (relative to the central longitudinal axis 122 of the frame 102). A “thickness” of a strut is measured between opposing locations on the radially facing inner and outer surfaces of a strut and is perpendicular to the width of the strut. In some examples, the width 144 of the axial struts 140 is 50-200%, 75-150%, or at least 100% larger than (e.g., double) the width of the angled struts of the frame 102.

By providing the axial struts 140 with the width 144 that is greater than the width of other, angled struts of the frame 102, a larger contact area is provided for when the leaflets 112 contact the wider axial struts 140 during systole, thereby distributing the stress and reducing the extent to which the leaflets 112 may fold over the axial struts 140, radially outward through the cells 118. As a result, a long-term durability of the leaflets 112 can be increased.

Since the cells 118 of the frame 102 can have a relatively large width compared to alternate prosthetic valves that have more than nine cells per row (as introduced above), the wider axial struts 140 can be more easily incorporated into the frame 102, without sacrificing open space for blood flow and/or coronary access.

Commissure tabs 115 of adjacent leaflets 112 can be secured together to form commissures 114 (FIG. 1). Each commissure 114 of the prosthetic heart valve 100 comprises two commissure tabs 115 paired together, one from each of two adjacent leaflets 112, and extending through a commissure window 142 of the frame 102. Each commissure 114 can be secured to the window struts 138 forming the commissure window 142.

The cusp edge portion (e.g., scallop edge) of each leaflet 112 can be secured to the frame 102 via one or more fasteners (e.g., sutures). In some examples, the cusp edge portion of each leaflet 112 can be secured directly to the struts of the frame 102 (e.g., angled struts 130, 132, and 134). For example, the cusp edge portions of the leaflets 112 can be sutured to the angled struts 130, 132, and 134 that generally follow the contour of the cusp edge portions of the leaflets 112.

In some examples, the cusp edge portion of the leaflets 112 can be secured to an inner skirt and the inner skirt can then be secured directly to the frame 102.

Various methods for securing the leaflets 112 to a frame, such as the frame 102, are disclosed in U.S. provisional patent applications 63/278,922, filed Nov. 12, 2021, and 63/300,302, filed Jan. 18, 2022, both of which are incorporated by reference herein.

As shown in FIGS. 2 and 3, in some examples, one or more of or each of the axial struts 140 can comprise an inflow end portion 146 (e.g., an end portion that is closest to the inflow end 108) and an outflow end portion 148 that are widened relative to a middle portion 150 of the axial strut 140 (which can be defined by the width 144). In some instances, the inflow end portion 146 of the axial strut 140 can comprise an aperture 147. The apertures 147 can be configured to receive fasteners (e.g., sutures) for attaching soft components of the prosthetic heart valve 100 to the frame 102. For example, in some instances, the outer skirt 106 can be positioned around the outer surface of the frame 102 and an upper or outflow edge portion of the outer skirt 106 can be secured to the apertures 147 by fasteners 149 (e.g., sutures), as shown in FIG. 1.

The interconnected struts 116 can also comprise horizontal struts 182 that extend between adjacent cells 118 of a row of cells of the frame 102 (FIGS. 2 and 3). The horizontal struts 182 can extend in a circumferential direction and also be referred to as circumferentially extending struts 182. The horizontal struts 182 can connect angled struts of two adjacent rows of angled struts of the frame 102 to one another. For example, each horizontal strut 182 can connect to two angled struts of one row of struts (for example, struts 134 shown in FIG. 3) and two angled struts in another, adjacent row of struts (for example, struts 132 shown in FIG. 3). As a result, an angled strut 184 extending between an axially extending window strut 138 and the horizontal strut 182 and an angled strut 186 extending between the horizontal strut 182 and another horizontal strut 182 disposed adjacent to the inflow end 108 of the frame 102 can be aligned along an angled line that can follow a scallop line of the leaflets (when the leaflets are attached to the frame 102). Thus, the horizontal struts 182 can allow the angled struts to follow a shape that more closely matches a shape of the scallop line of the leaflets when the frame 102 is in the radially expanded configuration (as shown in FIGS. 2 and 3). Additionally, the horizontal struts 182 can serve as spacers that can maintain a specified gap between the angled struts when the frame 102 is in the radially compressed configuration, thereby reducing a risk of pinching the leaflets between the struts in the radially compressed configuration.

The frame 102 can further comprise a plurality of apex regions 152 formed at the inflow end 108 and the outflow end 110, each apex region 152 extending and forming a junction between two angled struts 130 at the inflow end 108 or two angled struts 136 at the outflow end 110. As such, the apex regions 152 are spaced apart from one another, in a circumferential direction at the inflow end 108 and the outflow end 110.

Each apex region 152 can comprise an apex 154 (the highest or most outward extending, in an axial direction, point) and two thinned (or narrowed) strut portions 156, one thinned strut portion 156 extending from either side of the apex 154 to a corresponding, wider, angled strut 136 (at the outflow end 110) or angled strut 130 (at the inflow end 108) (FIG. 3). In this way, each of the apex regions 152 at the outflow end 110 can form a narrowed transition region between and relative to the two angled struts 136 extending from the corresponding apex region 152 and each of the apex regions 152 at the inflow end 108 can form a narrowed transition region between and relative to the two angled struts 130 extending from the corresponding apex region 152.

The thinned strut portions 156 of the apex regions 152 can have a width 158 that is smaller than a width 160 of the angled struts 130 or 136 (FIG. 3). In some examples, the width 158 can be a uniform width (e.g., along an entire length of the strut portion 156). In some examples, the width 158 of the thinned strut portions 156 can be from about 0.06-0.15 mm smaller than the width 160 of the angled struts 130 and/or 136.

The thinned strut portions 156 of the apex regions 152 can have a first length 162 (FIG. 3). In some examples, the first length 162 is in a range of 0.8-1.4 mm, 0.9-1.2 mm, 0.95-1.05 mm, or about 1.0 mm (e.g., ±0.03 mm). In alternate examples, the first length 162 is in a range of 0.3-0.7 mm, 0.4-0.6 mm, 0.45-0.55 mm, or about 0.5 mm (e.g., ±0.03 mm).

Thus, each outflow apex region 152 can include two thinned strut portions 156 having the first length 162, each extending from the apex 154, outward relative to a central longitudinal axis 164 of the cells 118. Thus, a total length of the apex region 152 can be two times the first length 162.

Each apex region 152 and two corresponding angled struts 136 at the outflow end 110 can form an outflow strut 166 and each apex region 152 and two corresponding angled struts 130 at the inflow end 108 can form an inflow strut 168.

Each outflow strut 166 and inflow strut 168 can have a length that includes an apex region 152 and the two angled struts 136 or 130 (or strut portions), respectively, on either side of the apex region 152. One half the total length of each outflow strut 166 and inflow strut 168 is shown in FIG. 3 as length 170, which extends from an end of one angled strut 136 or 130 to the central longitudinal axis 164. Thus, the length of each outflow strut 166 and inflow strut 168 is two times length 170. In some examples, the length 170 for half of each inflow strut 168 can be different than the length 170 for half of each outflow strut 166.

In some instances, the length of each thinned strut portion 156 can be at least 25% of the length 170 of the corresponding half outflow strut 166 or inflow strut 168. Said another way, the length of each apex region 152 (a total length being two times the first length 162) can be at least 25% of the total length (two times length 170) of the outflow strut 166 or inflow strut 168. In some examples, the length of each apex region 152 can be more than 25% of the total length of the corresponding outflow strut 166 or inflow strut 168, such as 25-35%.

In some examples, each apex region 152 can comprise a curved, axially facing outer surface 172 and an arcuate or curved, axially facing inner depression 174 which forms the thinned strut portions 156. For example, the curved inner depression 174 can depress toward the curved outer surface 172 from an inner surface of the angled strut portions 156, thereby forming the smaller width thinned strut portions 156. Thus, the curved inner depressions 174 can be formed on a cell side of the apex region 152 (e.g., as opposed to the outside of the apex region 152).

In some examples, the curved outer surface 172 of each apex region 152 can form a single, continuous curve from one angled strut portion 156 on a first side of the apex region 152 to another angled strut portion 156 on an opposite, second side of the apex region 152 (for example, the curved outer surface 172 can have a constant curvature).

Each apex region 152 can have a radius of curvature 176, along the curved outer surface 172 (e.g., in some instances, along an entirety or an entire length of the curved outer surface 172) (FIG. 3). In some instances, the radius of curvature 176 at the apex 154 and/or along the entire curved outer surface 172 of the apex region 152 can be greater than 1 mm. In some instances, the radius of curvature 176 can be in a range of 1-20 mm, 3-16 mm, or 8-14 mm. In some instances, the radius of curvature 176 can be greater than 10 mm. The radius of curvature 176 can be dependent on (and thus change due to changes in) the width 158 (e.g., the amount of reduction in width from the angled struts 130 or 136) and the first length 162 of the thinned strut portions 156.

Further, a height (an axial height) 178 of the apex regions 152, which can be defined in the axial direction from an outer surface of the two angled struts 130 or 136 to the curved outer surface 172 of the apex region 152 at the apex 154, can be the width 158 of the thinned strut portions 156 (FIG. 3). In this way, the height 178 of the apex regions 152 can be relatively small and not add much to the overall axial height of the radially expanded frame 102. Thus, the leaflets 112 secured to the frame 102 (FIG. 1) can be disposed close to the inflow end 108, thereby leaving a lar If ger open space at the outflow end 110 of the frame 102 that is not blocked by the leaflets 112.

In some examples, each of the apex region 152 can form an angle 180 between the two angled struts 130 or 136 extending from either side of the corresponding apex region 152 (FIG. 3). In some instances, the angle 180 can be in a range of 120 (not inclusive) to 140 degrees (e.g., such that the angle 180 is greater than 120 degrees and less than or equal to 140 degrees).

Additional details and examples of frames for prosthetic heart valves that include apex regions can be found in PCT Application No. PCT/US2022/025687, which is incorporated by reference herein.

FIG. 5 shows another exemplary prosthetic heart valve 350 (prosthetic valve). Similar to the prosthetic heart valve 100 of FIG. 1, the prosthetic valve 350 can have three main components: a stent or frame, 352, a valvular structure 354, and a sealing member 356.

The valvular structure 354 can comprise three leaflets 360, collectively forming a leaflet structure, which can be arranged to collapse in a tricuspid arrangement, although in other examples there can be greater or fewer number of leaflets (e.g., one or more leaflets 360). In some examples, the leaflets 360 can be formed of pericardial tissue (e.g., bovine pericardial tissue), biocompatible synthetic materials, or various other suitable natural or synthetic materials, the same or similar to the leaflets 112, as described above with reference to FIG. 1.

Each leaflet 360 can be coupled to the frame 352 along its inflow edge and at commissures 364 of the valvular structure 354 where adjacent portions (e.g., commissure tabs) of two leaflets are connected to each other. In some examples, the commissures 364 can comprise an attachment member 365 (e.g., comprising fabric, flexible polymer, or the like) arranged across a cell 374 (e.g., commissure cell) of the frame 352, the cell 374 formed by struts 372 (e.g., angled struts) of the frame 352. The attachment member 365 can be secured to the struts 372 of the frame forming the cell 374 and the adjacent portions of the two leaflets can be connected to the attachment member 365 to form the commissure 364.

In some examples, a reinforcing element or connecting skirt, such as a fabric strip, can be connected directly to the cusp edges of the leaflets and to the struts of the frame to couple the cusp edges of the leaflets to the frame.

The frame 352 can be made of any of various suitable plastically-expandable materials or self-expanding materials, such as any of the materials described herein with reference to the frame 102. When constructed of a plastically-expandable material, the frame 352 (and thus the prosthetic valve 350) can be crimped to a radially collapsed configuration on a delivery apparatus (e.g., the delivery apparatus 200 described below) and then expanded inside a patient by an inflatable balloon or equivalent expansion mechanism. Various crimping devices can be used to crimp the prosthetic valve 350 and the other prosthetic valves described herein around the delivery apparatus, such as the crimping devices described in U.S. Pat. No. 7,530,253, which is incorporated herein by reference.

In some instances, the prosthetic valve 350 can be crimped directly onto the inflatable balloon of the delivery apparatus, such that the prosthetic valve 350 is axially aligned with and disposed radially outward of the balloon during advancing the prosthetic valve on the delivery apparatus to the implantation site, for example as described in PCT Application No. PCT/US2021/047056, which is incorporated herein by reference. In alternate instances, the prosthetic valve 350 can be crimped onto the delivery apparatus axially offset from the balloon, and then moved over the balloon at the implantation site, prior to inflation of the balloon and radial expansion of the prosthetic valve, such as described in U.S. Pat. No. 9,339,384, which is incorporated herein by reference.

When constructed of a self-expandable material, the frame 352 (and thus the prosthetic valve 350) can be crimped to a radially collapsed configuration and restrained in the collapsed configuration by insertion into a sheath or equivalent mechanism of a delivery apparatus. Once inside the body, the prosthetic valve can be advanced from the delivery sheath, which allows the prosthetic valve to expand to its functional size.

The frame 352 in the illustrated example comprises a plurality of circumferentially extending rows of angled struts 372 defining rows of open cells 374 (or openings) of the frame. The frame 352 can have a cylindrical or substantially cylindrical shape having a constant diameter from the inflow end to the outflow end of the frame 352 as shown, or the frame 352 can vary in diameter along the height of the frame, as disclosed in U.S. Patent Publication No. 2012/0239142, which is incorporated herein by reference.

The frame 352, at each of the inflow end and the outflow end, may comprise a plurality of apices 380 spaced apart from one another around a circumference of the frame 352.

The sealing member 356 in the illustrated example is mounted on the outside of the frame 352 and functions to create a seal against the surrounding tissue (e.g., the native leaflets and/or native annulus) to prevent or at least minimize paravalvular leakage.

In some examples, the sealing member 356 can comprise an inner layer 376 (which can be in contact with the outer surface of the frame 352) and an outer layer 378. The sealing member 356 can be connected to the frame 352 using suitable techniques or mechanisms. For example, the sealing member 356 can be sutured to the frame 352 via sutures that can extend around the struts 372 and through the inner layer 376. In alternative examples, the inner layer 376 can be mounted on the inner surface of the frame 352, while the outer layer 378 is on the outside of the frame 352. The outer layer 378 can be configured or shaped to extend radially outward from the inner layer 376 and the frame 352 when the prosthetic valve 350 is deployed. When the prosthetic valve is fully expanded outside of a patient's body, the outer layer 378 can expand away from the inner layer 376 to create a space between the two layers. Thus, when implanted inside the body, this allows the outer layer 378 to expand into contact with the surrounding tissue.

Additional details regarding the prosthetic valve 350 and its various components are described in U.S. Patent Publication No. 2018/0028310, which is incorporated herein by reference.

In some examples, in lieu of the sealing member 356, the prosthetic heart valve 350 or any of the other prosthetic heart valves described herein (such as the prosthetic heart valve 100 of FIG. 1) can comprise an outer skirt or sealing member with one or more layers that are secured around an outer surface of the frame and sit closer to the outer surface of the frame (e.g., the frame 352 or the frame 102), such as described in PCT Application No. PCT/US2021/025045, which is incorporated herein by reference. For example, such an outer skirt can comprise a knitted outer layer and an inner layer (which can be woven, in some examples).

FIG. 4 shows a delivery apparatus 200, according to an example, that can be used to implant an expandable prosthetic heart valve (e.g., the prosthetic heart valve 100 of FIG. 1, the prosthetic heart valve 350 of FIG. 5, and/or any of the other prosthetic heart valves described herein). In some examples, the delivery apparatus 200 is specifically adapted for use in introducing a prosthetic valve into a heart.

The delivery apparatus 200 in the illustrated example of FIG. 4 is a balloon catheter comprising a handle 202 and a steerable, outer shaft 204 extending distally from the handle 202. The delivery apparatus 200 can further comprise an intermediate shaft 206 (which also may be referred to as a balloon shaft) that extends proximally from the handle 202 and distally from the handle 202, the portion extending distally from the handle 202 also extending coaxially through the outer shaft 204. Additionally, the delivery apparatus 200 can further comprise an inner shaft 208 extending distally from the handle 202 coaxially through the intermediate shaft 206 and the outer shaft 204 and proximally from the handle 202 coaxially through the intermediate shaft 206.

The outer shaft 204 and the intermediate shaft 206 can be configured to translate (e.g., move) longitudinally, along a central longitudinal axis 220 of the delivery apparatus 200, relative to one another to facilitate delivery and positioning of a prosthetic valve at an implantation site in a patient's body.

The intermediate shaft 206 can include a proximal end portion 210 that extends proximally from a proximal end of the handle 202, to an adaptor 212. A rotatable knob 214 can be mounted on the proximal end portion 210 and can be configured to rotate the intermediate shaft 206 around the central longitudinal axis 220 and relative to the outer shaft 204.

The adaptor 212 can include a first port 238 configured to receive a guidewire therethrough and a second port 240 configured to receive fluid (e.g., inflation fluid) from a fluid source. The second port 240 can be fluidly coupled to an inner lumen of the intermediate shaft 206.

The intermediate shaft 206 can further include a distal end portion that extends distally beyond a distal end of the outer shaft 204 when a distal end of the outer shaft 204 is positioned away from an inflatable balloon 218 of the delivery apparatus 200. A distal end portion of the inner shaft 208 can extend distally beyond the distal end portion of the intermediate shaft 206.

The balloon 218 can be coupled to the distal end portion of the intermediate shaft 206.

In some examples, a distal end of the balloon 218 can be coupled to a distal end of the delivery apparatus 200, such as to a nose cone 222 (as shown in FIG. 4), or to an alternate component at the distal end of the delivery apparatus 200 (e.g., a distal shoulder). An intermediate portion of the balloon 218 can overlay a valve mounting portion 224 of a distal end portion of the delivery apparatus 200 and a distal end portion of the balloon 218 can overly a distal shoulder 226 of the delivery apparatus 200. The valve mounting portion 224 and the intermediate portion of the balloon 218 can be configured to receive a prosthetic heart valve in a radially compressed state. For example, as shown schematically in FIG. 4, a prosthetic heart valve 250 (which can be one of the prosthetic valves described herein) can be mounted around the balloon 218, at the valve mounting portion 224 of the delivery apparatus 200.

The balloon shoulder assembly, including the distal shoulder 226, is configured to maintain the prosthetic heart valve 250 (or other medical device) at a fixed position on the balloon 218 during delivery through the patient's vasculature.

The outer shaft 204 can include a distal tip portion 228 mounted on its distal end. The outer shaft 204 and the intermediate shaft 206 can be translated axially relative to one another to position the distal tip portion 228 adjacent to a proximal end of the valve mounting portion 224, when the prosthetic valve 250 is mounted in the radially compressed state on the valve mounting portion 224 (as shown in FIG. 4) and during delivery of the prosthetic valve to the target implantation site. As such, the distal tip portion 228 can be configured to resist movement of the prosthetic valve 250 relative to the balloon 218 proximally, in the axial direction, relative to the balloon 218, when the distal tip portion 228 is arranged adjacent to a proximal side of the valve mounting portion 224.

An annular space can be defined between an outer surface of the inner shaft 208 and an inner surface of the intermediate shaft 206 and can be configured to receive fluid from a fluid source via the second port 240 of the adaptor 212. The annular space can be fluidly coupled to a fluid passageway formed between the outer surface of the distal end portion of the inner shaft 208 and an inner surface of the balloon 218. As such, fluid from the fluid source can flow to the fluid passageway from the annular space to inflate the balloon 218 and radially expand and deploy the prosthetic valve 250.

An inner lumen of the inner shaft can be configured to receive a guidewire therethrough, for navigating the distal end portion of the delivery apparatus 200 to the target implantation site.

The handle 202 can include a steering mechanism configured to adjust the curvature of the distal end portion of the delivery apparatus 200. In the illustrated example, for example, the handle 202 includes an adjustment member, such as the illustrated rotatable knob 260, which in turn is operatively coupled to the proximal end portion of a pull wire. The pull wire can extend distally from the handle 202 through the outer shaft 204 and has a distal end portion affixed to the outer shaft 204 at or near the distal end of the outer shaft 204. Rotating the knob 260 can increase or decrease the tension in the pull wire, thereby adjusting the curvature of the distal end portion of the delivery apparatus 200. Further details on steering or flex mechanisms for the delivery apparatus can be found in U.S. Pat. No. 9,339,384, which is incorporated by reference herein.

The handle 202 can further include an adjustment mechanism 261 including an adjustment member, such as the illustrated rotatable knob 262, and an associated locking mechanism including another adjustment member, configured as a rotatable knob 278. The adjustment mechanism 261 is configured to adjust the axial position of the intermediate shaft 206 relative to the outer shaft 204 (e.g., for fine positioning at the implantation site). Further details on the delivery apparatus 200 can be found in PCT Application No. PCT/US2021/047056, which is incorporated by reference herein.

As introduced above, in some examples, an outflow edge of an outer skirt and/or an inner skirt for a prosthetic heart valve can extend around a frame of the prosthetic heart valve at a location that is upstream of commissure support portions of the frame (which couple to commissures of the leaflet assembly of the prosthetic valve). For example, as depicted in FIG. 3, an outflow edge 190 of an outer skirt, depicted by a dashed line, extends around the frame 102 at a location upstream (toward the inflow end 108) of the axially extending window struts 138 (commissure support portions) which form the commissure windows 142. As a result, a gap 192 defined between the commissure windows 142 and the outflow edge 190 of the outer skirt (shown by distance 194 in FIG. 3) is formed. Blood can escape the prosthetic valve through the gaps 192, thereby resulting in perivalvular leakage and reduced efficiency of the prosthetic valve.

In some examples, the gaps 192 through which blood can pass through the frame 102 can be defined between inflow ends of the junctions 196 and the outflow edge 190 of the outer skirt (e.g., the open spaces in the cells). Downstream ends of the third row of angled struts 134 connect to each other at the junctions 196. A portion the junctions 196 connect to and/or form upstream (lower) ends of the axially extending window struts 138.

When commissures 114 are secured to the window struts 138, upstream edges of the commissures 114 are located at or adjacent to respective junctions 196 of the angled struts 134.

Another example of such a gap 382 is depicted in FIG. 5, the gap 382 defined between an outflow edge 358 of the sealing member 356 and strut junctions 375 formed between upper (or downstream) ends of a row of angled struts that are coupled to commissure support portions (angled struts 372 forming cells 374 across which the attachment member 365 extends) of the frame 352.

While positioning the outflow edge of the outer skirt upstream of the commissure support portions (toward the inflow end of the frame) can reduce a crimp profile of the prosthetic valve, such an arrangement also forms gaps between adjacent leaflets, adjacent to the commissure support portions of the frame, that are prone to perivalvular leakage (due to the frame not being covered by the outer skirt or leaflets in these gap regions).

In order to address perivalvular leakage through such gaps, while still maintaining a relatively small crimp profile for the prosthetic heart valve, an outer skirt for the prosthetic heart valve can comprise an outflow edge that forms circumferentially spaced apart projections that are aligned with the commissure support portions of the frame. The projections can extend from straight sections (also referred to as circumferentially extending sections) of the outflow edge of the outer skirt toward and/or to the commissure support portions of the frame, thereby covering the gaps or gap regions in the frame (as defined above). The straight sections can be disposed upstream of the commissure support portions of the frame.

For example, FIG. 6 shows an example of an outer skirt 400 comprising an outflow edge 402 and an inflow edge 404 disposed around an outer surface of a frame 102 of a prosthetic heart valve. Though the outer skirt 400 is shown disposed around the frame 102 in FIG. 6, the outer skirt 400 can be configured to be positioned around a variety of frames, such as the frame 352, or alternate frames for a prosthetic device.

In some examples, the outer skirt 400 is an annular skirt. In some instances, the outer skirt 400 can comprise one or more skirt portions that are connected together and/or individually connected to the frame 102. The outer skirt 400 can comprise a fabric or polymeric material, such as any of the materials described above with reference to the outer skirt 106 and/or the sealing member 356.

The outflow edge 402 forms a plurality of (e.g., three) circumferentially spaced projections 406 that are aligned with the commissure support portions of the frame 102 (e.g., the axially extending window struts 138 and commissure windows 142) when the outer skirt 400 is arranged around the frame 102. Since FIG. 6 depicts only a portion of the frame 102, including one commissure window 142, only one projection 406 is shown in FIG. 6. However, it should be noted that the outer skirt 400 can comprise three projections 406 that are spaced circumferentially apart from one another along the outflow edge 402. In examples where a frame includes more or less than three commissure support portions (e.g., commissure windows or frame cells), the outer skirt 400 can include a corresponding number of projections (e.g., two, four, or the like).

The outflow edge 402 further comprises straight sections 408 (which can also be referred to as circumferentially extending sections) extending between adjacent projections 406. For example, as shown in FIG. 6, the visible projection 406 is disposed between two straight sections 408. In some examples, the straight sections 408 can be completely straight and extend in a line from one projection 406 to the next projection 406. In some examples, the straight sections 408 can extend in a relatively straight path between adjacent projections 406 but can have some wave or curvature to the edge of the straight sections 408 (while still extending in a relatively straight path between adjacent projections 406).

As shown in FIG. 6, the straight sections 408 are located upstream of the junctions 196 of the second row of angled struts 134 (and thus upstream of the commissure support portions, or window strut struts 138 forming the commissure windows 142).

The projections 406 extend outwardly and away from the straight sections 408, toward the outflow end 110 of the frame (e.g., in a downstream direction). In some examples, the projections 406 can also be referred to as extensions of the outer skirt 400.

Each projection 406 is configured to cover a respective junction 196 of the second row of angled struts 134. In some examples, as shown in FIG. 6, a free end 410 (or downstream end or apex) of each projection 406 can extend toward the commissure window 142.

In some instances, the free end 410 of each projection 406 can extend to or over the commissure support portion of the frame, such as up to the inflow end of the commissure window 142 (over lower or inflow or upstream end portions of the axially extending window struts 138 and adjacent to an upstream edge of the commissure secured to the commissure window 142) of the frame 102 or up to or over a portion of cell of the frame configured to receive an attachment member of the commissure (e.g., the cell 374 of the frame 352 of FIG. 5).

Each projection 406 can be shaped to cover the downstream ends of a respective pair of angled struts of the third row of angled struts 134 and a respective junction 196. In some examples, as shown in FIG. 6, the shape of the projections 406 can be A-shaped or trapezoidal (an acute trapezoid that widens toward the straight sections 408).

In some examples, the edges of each projection 406 that extend between the free end 410 and the straight sections 408 can be more or less angled than shown in FIG. 6.

In some examples, the edges of each projection 406 can be curved to follow a contour of the angled struts 134 and extend over the respective junction 196. As a result, the projections can cover the gaps 192 (shown in FIG. 3) while still maintaining a reduced crimp profile of the radially compressed prosthetic valve.

Each projection 406 can be pre-formed in the outer skirt 400, such as being woven or knit with a remainder of the outer skirt 400. In some examples, each projection can be pre-formed in the outer skirt 400 by weaving and/or knitting a relatively rectangular outer skirt and then cutting sections out of the outflow end of the outer skirt 400 to form the projections 406 (e.g., prior to attaching the outer skirt 400 to the frame of the prosthetic valve).

The outflow edge 402 can be secured to the angled struts 134 and the junctions 196 with sutures, such as the stitches 512 and/or 514 shown in FIG. 7 (as described further below).

In some examples, as shown in FIG. 6, the inflow edge 404 of the outer skirt 400 can have cutouts 412 (spaces or indented regions) at the same circumferential position along the outer skirt 400 as the projections 406. For example, FIG. 6 shows one cutout 412 that is axially aligned with the projection 406.

Each cutout 412 can be shaped to conform to or follow the shape of the angled struts 130 defining the inflow end 108 of the frame 102 (e.g., the inflow struts 168). For example, as shown in FIG. 6, the cutout 412 can be shaped such that the inflow edge 404, at the region of the cutout 412, extends toward the outflow edge 402 from adjacent straight portions 414 (or sections) of the inflow edge 404 that are disposed on either side of the cutout 412. The cutout 412 is further shaped such that the inflow edge 404 extends along two adjacent and connected angled struts 130 that form two adjacent inflow struts 168. The cutout 412 can have an apex or peak 416 at a junction (e.g., horizontal struts 182) between the two angled struts 130 and two angled struts 132 of the adjacent row of angled struts 132.

The cutouts 412 allow an overall height (measured in the axial direction) of the outer skirt 400 to remain substantially the same along an entire circumference of the outer skirt 400. For example, as shown in FIG. 6, a first height 418 that is defined between the peak 416 of the cutout 412 and the free end 410 of the projection 406 can be the same or substantially the same as a second height 420 that is defined between the straight section 408 of the outflow edge 402 and the straight portion 414 of the inflow edge 404.

Preserving substantially the same height along the outer skirt 400 can be advantageous to retain a reduced (or minimized) crimp profile of the radially compressed prosthetic valve.

FIG. 7 shows another example of an outer skirt 500 comprising an outflow edge 502 and an inflow edge 504 disposed around an outer surface of a frame 102 of a prosthetic heart valve. Though the outer skirt 500 is shown disposed around the frame 102 in FIG. 7, the outer skirt 500 can be configured to be positioned around a variety of frames, such as the frame 352, or alternate frames for a prosthetic device.

In some examples, the outer skirt 500 is an annular skirt. In some instances, the outer skirt 500 can comprise one or more skirt portions that are connected together and/or individually connected to the frame 102.

The outer skirt 500 can comprise a fabric or polymeric material, such as any of the materials described above with reference to the outer skirt 106 and/or the sealing member 356.

In some examples, the outer skirt 500 comprises a stretchable fabric or polymeric material, such as thermoplastic polyurethane (TPU), or the like.

In some examples, the outer skirt 500 can comprise one or more fabric or polymeric materials that are knitted together (or some other non-woven structure) to form the outer skirt 500. In some instances, such materials can exhibit at least a minimal recoverable strain. Such knitted or non-woven outer skirts can have elasticity (be configured to stretch) due to the filaments of the material bending.

Thus, when the outer skirt 500 is arranged around the outer surface of the frame 102, as shown in FIG. 7, portions of the outflow edge 502 that are aligned with the commissure support portions of the frame 102 (e.g., axially extending window struts 138 and commissure windows 142) can be stretched toward the lower (or inflow) end of the commissure windows 142, over the junctions 196, and secured to the frame 102 (e.g., with one or more stitches 514, as described further below). As a result, a plurality of (e.g., three) circumferentially spaced projections 506 are formed in the outflow edge 502, with straight sections 508 formed between adjacent projections 506. The projections 506 cover gaps between the junctions 196 and the straight sections 508 of the outer skirt 500 that were present prior to the stretching of the portions of the outflow edge 502. The straight sections 508 can be the same or similar to the straight sections 408, as described above.

In some examples, the outer skirt 500 (and the outer skirt 400) can be attached to the frame 102 along the angled struts to which the cusp edge portions (scallop line) of the leaflets are attached. For example, as shown in FIG. 7, the outer skirt 500 is attached to angled struts 134, 132, and 130 that follow a scallop line of the leaflets (when the leaflets are secured on an inside of the frame 102) by stitches 512 (with one or more sutures). In some examples, an additional stitch 514 (or one or more additional stitches) can attach each projection 506 to a respective commissure support portion and/or junction 196. In some examples, the stitch 514 can extend around the junction 196, through the commissure window 142, and through a material of the outer skirt 500 (at the projection 506).

The projections 506 (one shown in FIG. 7) can be similar to the projections 406, as described above, in that they cover respective junctions 196 of the second row of angled struts 134 when secured to the frame 102. In some examples, as shown in FIG. 7, free end 510 (or downstream end) of each projection 506 can extend toward and/or up to the commissure support portion of the frame, such as up to the inflow end of the commissure window 142 (over lower or inflow or upstream end portions of the axially extending window struts 138) of the frame 102 or up to or over a portion of cell of the frame configured to receive an attachment member of the commissure (e.g., the cell 374 of the frame 352 of FIG. 5).

As shown in FIG. 7, the straight sections 508 are located upstream of the junctions 196 of the second row of angled struts 134 (and thus upstream of the commissure support portions, or window strut struts 138 forming the commissure windows 142). The projections 506 extend outwardly and away from the straight sections 508, toward the outflow end 110 of the frame (e.g., in a downstream direction). In some examples, the projections 506 can also be referred to as extensions of the outer skirt 500.

When secured to the frame 102, as shown in FIG. 7, each projection 506 can cover the downstream ends of a respective pair of angled struts of the third row of angled struts 134 and a respective junction 196. In some examples, the resulting shape of the projections 506 can be A-shaped or trapezoidal (an acute trapezoid that widens toward the straight sections 508).

In some examples, as shown in FIG. 7, the inflow edge 504 of the outer skirt 500 can be relatively straight. In some instances, the straight sections 508 of the outflow edge 502 can extend in parallel to the inflow edge 504.

In some examples, the inflow edge 504 of the outer skirt 500 can be wavy and/or follow a shape of the inflow struts 168.

The outer skirt 500 has a first height 516 defined between the straight sections 508 of the outflow edge 502 and the inflow edge 504. By stretching the outflow edge 502 of the outer skirt 500 to form the projections 506, the outer skirt 500 has a second height 518 at the projections 506 and regions of the commissure support portions of the frame 102 when secured to the frame 102 as shown in FIG. 7, the second height 518 larger than the first height 516. The second height 518 can be measured between the free end 510 of the projection 506 and the inflow edge 504 of the outer skirt 500.

In some examples, instead of an outer skirt with projections, attachment members for commissures of the prosthetic valve can have extensions configured to cover the gaps or gap regions in the frame (as described above and depicted in FIGS. 3 and 5). For example, FIGS. 8-11 show an attachment member 600 (FIGS. 8-10) or attachment member 650 (FIG. 11) that is configured to form a commissure with adjacent edges or ends (e.g., commissure tabs) of the leaflets of the prosthetic valve and attach to a commissure support portion of the frame of the prosthetic valve. The attachment member can comprise a flexible fabric, flexible polymer, or the like.

Though a single attachment member 600 and commissure 640 formed with the attachment member 600 are shown in FIGS. 8-10, it should be noted that the prosthetic valve can have a plurality of such attachment members 600 and commissures 640 (e.g., three if the prosthetic valve has three leaflets). Additionally, although the attachment member 600 is described below and shown in FIG. 10 as being configured to attach to commissure windows in the frame 102 and cover a gap in the frame 102, it should be noted that the attachment member 600, and/or similar attachment members, can be adapted to be used with a variety of prosthetic valve frames, such as frames having differently shaped commissure windows and/or frames where the commissure support portions are formed by struts forming cells of the frame (e.g., frame 352 of FIG. 5).

As shown in FIG. 8, the attachment member 600 (also referred to herein in some examples as a commissure extension member or patch) comprises a main body 602 (or upper portion) and an extension 604 that extends outward and away from a lower lateral edge 606 (or inflow edge) of the main body 602. As described further below, the main body 602 of the attachment member 600 can be configured to attach to a commissure support portion of a frame of a prosthetic valve and the extension 604 can be configured to extend axially upstream of the commissure support portion to cover a gap between an outflow edge of the outer skirt of the prosthetic valve and the commissure support portion.

In some examples, the attachment member 600 does not need to perform an attachment function for attaching commissures to the frame of the prosthetic valve. Instead, in some instances, the attachment member 600 could simply serve to cover the gap in the frame via the extension 604. In such examples, the attachment member 600 could extend between a commissure and the outer skirt (without attaching the commissure to the frame).

In some examples, as shown in FIGS. 8-10, the main body 602 can comprise two laterally extending flaps 608 and the extension 604 can extend outward from a central portion of the main body 602 where the two flaps 608 connect together. A shape and size of the main body 602 can be configured to couple with the commissure support portion of the frame.

For example, in some instances, a height 610 of the main body 602 and the flaps 608 can be specified to fit through a commissure window 142 of the frame 102 (as shown in FIG. 10). Specifically, in some instances, the height 610 can be similar to or slightly smaller than a height of the commissure window 142 (the heights defined in the axial direction of the prosthetic valve).

In some instances, a length 612 of each of the flaps 608 can be specified such that each flap 608 can align with a commissure tab of a leaflet of the prosthetic valve and be folded with the commissure tab and around an axially extending window strut 138 of the frame, as described further below. As shown in FIG. 8, the length 612 of each flap 608 can be defined between a free edge 614 of the flap 608 and a central axis 616 of the attachment member 600.

In some examples, the free edges 614 of the flaps 608 are parallel to one another (as shown in FIG. 8).

In some examples, the free edges 614 of the flaps 608 are perpendicular to the lower lateral edge 606 and an upper lateral edge 618 of the main body 602.

As shown in FIG. 10 and introduced above, the outflow edge 190 of the outer skirt 106 of the prosthetic valve can extend around the frame 102 at a location that is offset from and upstream of the junctions 196, thereby forming an outer skirt having a height 642. Thus, the extension 604 of the attachment member 600 is shaped to cover a gap in the frame 102 disposed between the outflow edge 190 of the outer skirt 106 (or an alternate outer skirt disposed around the frame) and the respective junction 196.

In some examples, the extension 604 can be an axially extending extension that tapers from a free end 620 (or upstream end) of the extension 604 to a downstream end 622 of the extension 604 which connects to the main body 602. For example, the extension 604 can have two angled edge 624 that angle inward, toward one another, from the free end 620 to respective flaps 608.

In some examples, the extension 604 has a trapezoidal shape that widens toward the free end 620 (as shown in FIG. 6).

In some examples, the extension 604 can be V-shaped, with the V wide enough, or the point of the V connected low enough on the outer skirt, such that the gap between the respective junction 196 and outflow edge 190 is covered when the prosthetic valve is in its radially expanded configuration.

To couple the attachment member 600 to the frame 102 (e.g., to the axially extending window struts 138 forming the commissure window 142), in some examples, each flap 608 can be folded over itself (toward the opposite flap 608) on the radially outward facing surface of the axially extending window strut 138 (FIG. 9). The flap 608 then folds radially inward to extend through the commissure window 142 and across the laterally inward facing surface of the axially extending window strut 138 (FIG. 9). The flap 608 is then folded over the radially inward facing surface of the axially extending window strut 138, away from the opposite flap 608 (FIG. 9).

As used herein, the radially inward facing and radially outward facing surfaces of the axially extending window struts (or another component of the prosthetic valve) are relative to a central longitudinal axis of the prosthetic valve (e.g., central longitudinal axis 122 of FIG. 1).

In some examples, adjacent ends of two adjacent leaflets, such as adjacent commissure tabs, can be arranged against respective flaps 608 of the attachment member 600 and then folded together with the flaps 608 around the axially extending window struts 138, as described above. For example, in some instances, when the leaflets are arranged within an interior of the frame 102, adjacent commissure tabs can be pushed through a respective commissure window 142 to an exterior of the frame, and then paired with respective flaps 608 and folded together with the flaps 608, as described above.

After folding the flaps 608 around the axially extending window struts 138, the folds of the flaps 608 can then be sutured together around the axially extending window struts 138.

In some instances, the flaps 608 can be further sutured to the commissure tabs of the two adjacent leaflets, thereby forming a commissure 640 (FIG. 10). It should be noted that the leaflets are omitted from FIGS. 9-11 for the purposes of illustration alone, and that the final commissure 640 would include adjacent ends (e.g., commissure tabs) of two adjacent leaflets secured to the folded flaps 608 of the attachment member 600.

The extension 604 of the attachment member can then be arranged over the gap region (between the junction 196 and the outflow edge 190 of the outer skirt 106) and secured to the frame 102 and/or the outflow edge 190 of the outer skirt 106, thereby sealing the gap or gap region with the extension 604. As a result, perivalvular leakage through the frame 102 is reduced when the prosthetic valve is implanted in the native anatomy at the implantation site.

For example, as shown in FIG. 10, the free end 620 of the extension 604 can be attached to the outflow edge 190 of the outer skirt 106 with one or more stitches 626 (sutures). In some instances, the extension 604 can be secured to the frame, such as to portions of the angled struts 134 that extend between the outer skirt 106 and the junctions 196.

In some instances, the free end 620 of the extension 604 overlaps the outflow edge 190 and is disposed on an outer surface of the outer skirt 106 (such that the outflow edge 190 is disposed between the free end 620 and the frame 102).

In some instances, the free end 620 of the extension 604 overlaps the outflow edge 190 and is disposed on an inner surface of the outer skirt 106 (such that the free end 620 is disposed between the outflow edge 190 and the frame 102).

In some examples, instead of having laterally extending flaps 608 that fold and extend through a commissure window 142 of the frame 102, a main body of an attachment member can be configured to fit across a cell of the frame of the prosthetic valve, such as a cell 374 of the frame 352 shown in FIG. 5. The extension of the attachment member can then extend upstream, toward the outflow edge of the outer skirt to cover a gap in the frame (e.g., gap 382) between struts junctions 375 and the outflow edge.

In some examples, a commissure attachment member can comprise two separate attachment member portions (one for each commissure tab of the two adjacent leaflets). For example, as shown in FIG. 11, an attachment member 650 can comprise a first attachment member portion 652a and a second attachment member portion 652b, each comprising a main body 654a, 654b and an extension 658a, 658b, the main body 654a, 654b including a laterally extending flap 656a, 656b. The components of the attachment member portions 652a, 652b can be configured the same or similar to the corresponding components of the attachment member 600 (e.g., the flaps, extensions, and the like), except the main bodies 654a, 654b and extensions 658a, 658b are half portions (or asymmetric divided portions, such as ⅓, ⅔) of the main body 603 and extension 604 of the attachment member 600.

In some examples, the first and second attachment member portions 652a, 652b are equal halves of the whole attachment member 650 (as depicted in FIG. 11 by dividing line 660).

In some examples, the first and second attachment member portions 652a, 652b are unequal halves of the whole attachment member 650, such as one of the first and second attachment member portions 652a, 652b being larger than the other one of the first and second attachment member portions 652a, 652b.

Each of the first and second attachment member portions 652a, 652b can be paired with and attached to a corresponding upper portion (e.g., commissure tab) of a leaflet (of two adjacent leaflets), prior to assembly within the frame of the valve. Then, when the leaflets are disposed within the frame (e.g., frame 102), each of the first and second attachment member portions 652a, 652b can be pushed radially outward through the commissure window (e.g., window 142) and then folded and attached to respective axially extending window struts, in the same manner as described above with reference to FIGS. 9 and 10. Following this attachment to the frame, the first and second attachment member portions 652a, 652b can then be secured (e.g., sutured) together along the dividing line 660.

By using two separate attachment members (or attachment member portions) for each leaflet of a commissure, the attachment members or attachment member portions can be more easily attached to the leaflets outside the frame (e.g., in a flat configuration) prior to attachment to the frame, thereby simplifying the prosthetic valve assembly process.

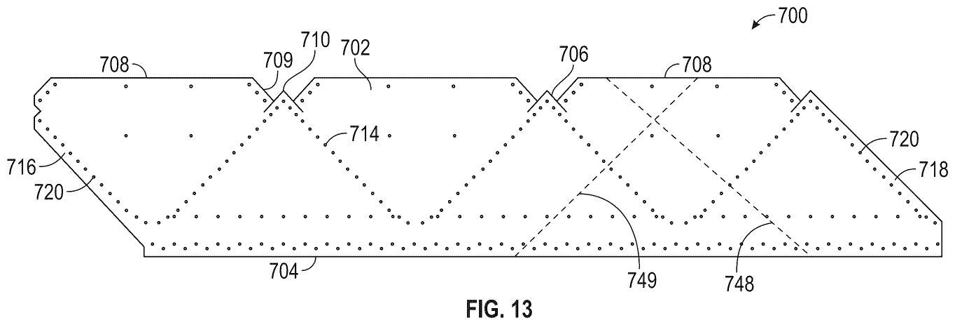

In some examples, instead of, or in addition to, an outer skirt with projections, the prosthetic valve can have an inner skirt with circumferentially spaced apart projections that are aligned with the commissure support portions of the frame, where the projections are configured to cover the gaps or gap regions in the frame (similar to as described above and depicted in FIGS. 3 and 5). For example, FIGS. 12 and 13 show an example of an inner skirt 700 comprising an outflow edge portion 702 and an inflow edge portion 704. FIG. 13 shows the inner skirt 700 in a straightened (non-annular) configuration, and FIG. 12 shows the inner skirt 700 disposed around an inner surface of a frame 102 of a prosthetic heart valve (and FIG. 16 shows a complete side view of the inner skirt 700 disposed around the frame 102 in an annular configuration). Though the inner skirt 700 is shown disposed around the frame 102 in FIGS. 12 and 16, the inner skirt 700 can be configured to be positioned around a variety of frames, such as the frame 352, or alternate frames for a prosthetic device.

In some examples, the inner skirt 700 is an annular skirt (as shown in FIGS. 12 and 13).

In some instances, the inner skirt 700 can comprise one or more skirt portions that are connected together and/or individually connected to the frame 102.

The inner skirt 700 can comprise a fabric or polymeric material, such as any of the skirt materials described herein.

The outflow edge portion 702 forms a plurality of (e.g., three) circumferentially spaced projections 706 (as shown in FIG. 13) that are aligned with the commissure support portions of the frame 102 (e.g., the axially extending window struts 138 and commissure windows 142) when the inner skirt 700 is arranged around the frame 102 (as shown in FIG. 12). Since FIG. 12 depicts only a portion of the frame 102, including one commissure window 142, only one projection 706 is shown in FIG. 12. However, as shown in FIG. 13, the inner skirt 700 can comprise three projections 706 that are spaced circumferentially apart from one another along the outflow edge portion 702. In examples where a frame includes more or less than three commissure support portions (e.g., commissure windows or frame cells), the inner skirt 700 can include a corresponding number of projections (e.g., two, four, or the like).

The outflow edge portion 702 further comprises straight sections 708 (which can also be referred to as circumferentially extending sections) extending between adjacent projections 706. For example, as shown in FIG. 12, the visible projection 706 is disposed between two straight sections 708.

In some examples, as shown in FIG. 13, the straight sections 708 can be disposed above (and axially offset from) the projections 706 when the inner skirt 700 is not coupled to the frame. In such a configuration, the straight sections 708 can, in some examples, be directly connected to the projections 706 by angled portions 709 (or edges). During or prior to arranging the inner skirt 700 around the inner surface of the frame 102, the straight sections 708 can be folded over to form a folded edge 711 of the straight sections 708 (as shown in FIG. 12, and also depicted in FIG. 17 which is described in further detail below). The angled portions 709 enable the folded edge 711 to connect between the adjacent projections 706 and form an outflow edge of the inner skirt 700 that is disposed upstream of the projections 706 (as shown in FIG. 12). Thus, the folded edge 711 of the straight sections 708 can also be referred to as the straight sections of the outflow edge of the inner skirt 700, when the inner skirt 700 is arranged around the frame 102.

In some examples, the inner skirt 700 may not include straight sections 708 that are configured to be folded over the inner skirt 700, and instead the inner skirt 700 may include an outflow edge portion 702 with straight sections that are disposed below (or upstream) of the projections 706 both prior to and after arranging the inner skirt 700 around the frame 102.

As shown in FIG. 12, the folded edges 711 of the straight sections 708 are located upstream of the junctions 196 of the second row of angled struts 134 (and thus upstream of the commissure support portions, or window strut struts 138 forming the commissure windows 142).

The projections 706 extend outwardly and away from the (folded over) straight sections 708, toward the outflow end 110 of the frame 102 (e.g., in a downstream direction). In some examples, the projections 706 can also be referred to as extensions of the inner skirt 700.

Each projection 706 is configured to cover a gap in the frame that is formed between a respective junction 196 and the folded edges 711 of the straight sections 708 of the inner skirt 700. In some examples, as shown in FIG. 12, a free end or apex 710 of each projection 706 can extend toward the commissure window 142.

Though FIG. 12 depicts the apex 710 of the projection 706 extending to an upstream end of the junction 196, in some examples the apex 710 of the projection 706 can extend over a surface of the junction 196 and/or upstream end portions of the axially extending window struts 138, on an inside of the frame (behind the frame in the view of FIG. 12).

In some examples, each projection 706 can be shaped to cover or be arranged flush against the edges of the downstream ends of a respective pair of angled struts of the third row of angled struts 134 and a respective junction 196. In some examples, as shown in FIGS. 12 and 13, the shape of the projections 706 can be A-shaped or trapezoidal (an acute trapezoid that widens toward the folded edges 711 of the straight sections 708).

In some examples, the edges of each projection 706 that extend between the apex 710 and the folded edges 711 of the straight sections 708 can be more or less angled than shown in FIGS. 12 and 13.

As shown in FIG. 13, each projection 706 can be pre-formed in the inner skirt 700, such as being woven or knit with a remainder of the inner skirt 700.

FIG. 16 shows a prosthetic valve including the inner skirt 700 secured to the frame 102 (on an inner surface of the frame 102), leaflets 112 disposed within the frame and forming commissures 114 secured to the commissure windows 142, and an exemplary outer skirt 750 disposed around an outer surface of the frame 102.

The outflow edge portion 702 of the inner skirt 700 can be secured to the angled struts 134 and the junctions 196 with sutures, such as the stitches 712 shown in FIG. 16.

In some examples, the stitches 712 can extend through pre-formed apertures 714 in the inner skirt 700 (as shown in FIG. 13). In some examples, the apertures 714 and stitches 712 can follow the cusp edge portions of the leaflets. For example, the apertures 714 can, as shown in FIG. 13, undulate from the first edge portion 716 to the second edge portion 718, thereby following a shape of the cusp edge portions of the leaflets.

In some examples, the stitches 712 can also secure the cusp edge portions of the leaflets to the inner skirt 700, thereby securing the leaflets to the frame 102.

In some examples, separate stitches (such as stitches 724 shown in FIG. 18) can secure the leaflets to the inner skirt and the stitches 712 can secure the inner skirt 700 to the frame (for example by looping through the stitches 724 and around the frame struts, or by extending the stitches 712 through the skirt material and then around the frame struts).

As shown in FIG. 16, in some examples, an outflow edge 752 of the outer skirt 750 can be offset from and disposed upstream of the corresponding outflow edge portion 702 (or outflow edge) of the inner skirt 700, and an inflow edge 754 of the outer skirt 750 can be offset from and disposed downstream of the corresponding inflow edge portion 704 (or inflow edge) of the inner skirt 700.

In some examples, the outflow edge 752 and the inflow edge 754 of the outer skirt 750 can be secured to the inner skirt 700 and/or the frame 102 with stitches 756, as shown in FIG. 16.

In this way, by utilizing an outer skirt and/or an inner skirt with projections formed along its outflow edge and aligned with commissure support portions of the frame (as described above with reference to FIGS. 6 and 7, and FIGS. 12, 13, and 16), or commissure attachment members with extensions extending in an upstream direction, gaps or openings in the valve frame disposed between the commissure support portions and the outflow edge of the skirt(s) (e.g., the relatively straight portions of the outflow edge) can be covered, thereby providing increased perivalvular leakage (PVL) sealing. At the same time, by allowing a majority or the relatively straight portions of the outflow edge of the skirt(s) to be positioned upstream of the commissure support portions, a reduced crimp profile of the radially compressed prosthetic heart valve can be maintained. As such, the skirt(s) and/or attachment member configurations described herein provide for both a reduced crimped profile and increased PVL sealing of a prosthetic heart valve.

In some examples, an inner skirt can have angled edge portions that are configured to overlap when the inner skirt is arranged in an annular configuration and secured on an inside of the frame of the prosthetic valve. A cusp edge portion of one leaflet of the prosthetic heart valve can be arranged along the overlapping edge portions of the inner skirt and all three overlapping layers (two skirt layers and one leaflet layer) can be secured together (for example, with stitches). This can simplify assembly of the prosthetic valve by reducing assembly time and reducing an amount of material that is used in the assembly process.

For example, the inner skirt 700 shown in FIG. 13 has a first edge portion 716 and a second edge portion 718 which each extend between the inflow edge portion 704 and the outflow edge portion 702. The first and second edge portions 716, 718 can be non-perpendicular to the inflow edge portion 704. For example, the first and second edge portions 716, 718 can extend at non-zero angles, such as at angles of about 45 degrees (or in a range of 40 to 50 degrees) relative to the inflow edge portion 704 (and/or the straight sections 708 of the outflow edge portion 702). The first edge portion 716 and the second edge portion 718 can be referred to as angled edges of the inner skirt 700.

The first and second edge portions 716, 718 can each include a line of spaced apart apertures 720 (or perforations) that extend through a material of the inner skirt 700. When the inner skirt 700 is converted into its annular configuration (for example, when mounted around a prosthetic device, as shown in FIGS. 12 and 16 for example), the first and second edge portions 716, 718 can overlap one another with their respective apertures 720 overlapping as well (as shown in FIGS. 14 and 15). A cusp edge portion 113 of a leaflet 112 can be arranged against the overlapping first and second edge portions 716, 718 (e.g., as shown in FIG. 14 where the leaflet is disposed behind the inner skirt 700 in the view of FIG. 14). In some examples, the cusp edge portion 113 of the leaflet 112 can also include apertures that align with the apertures 720. A suture can then be used to form a plurality of stitches in and in-and-out pattern through the overlapping apertures 720 (e.g., stitches 722 shown in FIG. 14) and the cusp edge portion 113 of the leaflet 112 (or the apertures formed in the cusp edge portion), thereby securing the first and second edge portions 716, 718 and the leaflet 112 together and forming the annular configuration of the inner skirt 700.

In some examples, as shown in FIG. 13, each of first and second edge portions 716, 718 includes only a single line of apertures 720, and the single line of apertures 720 can be used to secure the overlapping first and second edge portions 716, 718 and the cusp edge portion of the leaflet 112 to one another with a single line of in-and-out stitches 722, as shown in FIG. 14. This can simplify the assembly process by decreasing assembly time and materials.

As shown in FIG. 15, the in-and-out stitches 722 can extend through the overlapping apertures 720 and the cusp edge portion of the leaflet 112. In some examples, as shown in FIG. 15, the in-and-out stitches 722 can also extend through apertures in the cusp edge portion that overlap with the apertures 720 in the first and second edge portions 716, 718.

During assembly of the prosthetic valve, a remainder of the cusp edge portion of the leaflet 112 shown in FIG. 14 can be secured to additional apertures 714 in the inner skirt 700 by stitches 724 shown in FIG. 18. For example, FIG. 18 depicts another portion of the inner skirt 700 and leaflet 112 disposed underneath (or behind in the view of FIG. 18) the inner skirt 700 with its cusp edge portion aligned along the line of apertures 714 and stitches 724 extending therethrough, thereby securing the cusp edge portion of the leaflet 112 to the inner skirt 700. All the leaflets 112 of the prosthetic valve can be secured to the inner skirt 700 in a similar manner, thereby forming a leaflet assembly secured to the inner skirt 700.

The inner skirt 700 can be secured to the frame with sutures. In some examples, the stitches 712 shown in FIG. 16 can extend through the stitches 724 or through a material of the inner skirt 700 to secure the inner skirt 700 to the frame 102.

In some examples, instead of separate stitches, the same stitches (such as stitches 724) can be used to secure the leaflets to the inner skirt and the inner skirt to the frame.

In some examples, the outflow edge portion 702 of the inner skirt 700, the inflow edge portion 704 of the inner skirt 700, or both the outflow edge portion 702 and the inflow edge portion 704 can be folded over itself or themselves such that free edges of the outflow edge portion 702 and/or inflow edge portion 704 are facing outwards, away from the leaflets of the prosthetic valve.

For example, FIG. 17 shows a portion of the inner skirt 700 with the outflow edge portion 702 folded over itself onto an outer surface 726 of the inner skirt 700 such that a free edge of the outflow edge portion 702 is arranged against the outer surface 726. The outer surface 726 can be the surface of the inner skirt 700 that faces and is arranged against an inner surface of the frame when the inner skirt 700 is disposed around the frame. As such, the outer surface 726 can be referred to as a radially outward facing surface relative to a central longitudinal axis of the prosthetic valve.

In some examples, as introduced above, each straight section 708 of the inner skirt 700 can be folded over to form a plurality of folded portions 728, each with a folded edge 711, that are spaced apart around the inner skirt 700 (for example, by the projections 706) (as shown in FIGS. 17 and 18). The folded portions 728 of the outflow edge portion 702 can be secured to a body of the inner skirt by sutures, such as with stitches 730, as shown in FIGS. 17 and 18.

By folding over the outflow edge portion 702 in this way, unwanted abrasion of the leaflets against the free edge of the outflow edge portion 702 can be reduced, thereby increasing the long-term durability of the leaflets.

In some examples, the folded portions 728 of the outflow edge portion 702 of the inner skirt 700 can be arranged on an inside of the frame (against the inner surface of the frame 102, for example, as shown in FIG. 16).

In some examples, the folded portions 728 (prior to securing to the body of the inner skirt 700) can extend through the frame (e.g., through cells of the frame) and be folded over a corresponding outflow edge of the outer skirt, on an exterior of the frame.

In some examples, the inflow edge portion 704 can also extend through the frame and be folded over a corresponding inflow edge of the outer skirt.

For example, FIG. 19 shows the outflow edge portion 702 of the inner skirt 700 forming a plurality of folded portions 728 extending through cells 118 of the frame 102 and folded over (and covering) the outflow edge 752 of the outer skirt 750. FIG. 19 also shows the inflow edge portion 704 of the inner skirt 700 extending around the inflow end 108 of the frame 102 and folded over (and covering) the inflow edge 754 of the outer skirt 750, thereby forming a folded portion 732 of the inflow edge portion 704.

In some examples, the outer skirt 750 can comprise a plurality of outwardly extending fibers or filaments which can be configured to encourage tissue ingrowth (for improved sealing against the native anatomy). By folding the inner skirt 700 over the inflow and outflow edges (or edge portions) of the outer skirt 750, the outwardly protruding fibers of the outer skirt 750 (or another relatively rough material of the outer skirt) can be prevented from extending inward into the frame and coming into contact with the leaflets and/or can prevent unwanted tissue overgrowth along the outflow and inflow edges of the outer skirt 750.

In some examples, in order to facilitate the outflow edge portion 702 of the inner skirt 700 extending through the cells 118 of the frame and around the axial struts 140 and/or axially extending window struts 138 (as shown in FIG. 19), the outflow edge portion 702 can include slits 734 (or cuts) that extend through the inner skirt 700, as shown in FIG. 20. For example, the outflow edge portion 702 can include two pairs of angled slits 734 in each straight section 708, thereby forming three folded portions 728 in each straight section 708. Each folded portion 728 is configured to extend through a respective cell 118 of the frame, with adjacent folded portions 728 extending on either side of an axial struts 140 or axially extending window strut 138 (as shown in FIG. 19).

In some examples, instead of being angled, as shown in FIG. 20, the slits 734 can be perpendicular to the free edge of the straight sections 708.

In some examples, the inner skirt 700 can include more or less slits 734 than shown in FIG. 20. For example, a single slit 734 can be used between each folded portion 728 or more or less pairs of slits 734 can be formed depending on the number of cells in each row of cells of the frame.

In some examples, as shown in FIG. 20, the outflow edge portion 702 can include a plurality of spaced apart apertures 738 disposed therethrough. A portion of the inner skirt 700 disposed away from the outflow edge portion 702 and toward the inflow edge portion 704 can include a plurality of spaced apart apertures 740 disposed therethrough. When the straight sections 708 are folded over, as described above, the apertures 738 and apertures 740 can overlap one another and then sutures can be used to secure the folded portions 728 to the body of the inner skirt 700, such as with a plurality of in-and-out stitches 742 (as shown in FIG. 21).

In some examples, as shown in the cross-sectional view of FIG. 21, stitches 742 can extend through the outer skirt 750 as well.

It should be noted that the arrangement of apertures 738 and 740 shown in FIG. 20 is exemplary, and other numbers and arrangements of apertures for securing the folded portions 728 around the frame and to the inner skirt 700 are possible. In some examples, the inner skirt 700 may not include pre-formed apertures 738 and 740, and instead apertures can be formed in the folded portions 728 and the body of the inner skirt 700 as a needle pierces the fabric of the inner skirt 700 and forms the in-and-out stitches.

In some examples, as shown in FIG. 20 (and FIG. 13), the inflow edge portion 704 can include a plurality of apertures 736. When the inflow edge portion 704 is folded over itself to form the folded portion 732, in-and-out stitches 744 can be formed through the apertures 736, thereby securing the folded portion 732 to the body of the inner skirt 700 (and/or to the outer skirt 750). For example, FIG. 21 shows an exemplary stitch 744 extending through the folded portion 732 of the inflow edge portion 704, the outer skirt 750, and the body of the inner skirt 700 on an inside of the frame.

As shown in FIG. 21, in some examples, the straight sections 708 of the outflow edge portion 702 of the inner skirt 700 can be folded such that a portion of each is folded over itself on the inside of the frame 102 (creating inner folded portion 746), and then another portion extends through the frame 102 and over an outer surface of the outer skirt 750 (creating the folded portion 728).

Returning to FIG. 13, in some examples, the inner skirt 700 can comprise a plurality of first filaments 748 and a plurality of second filaments 749 that extend at 45-degree angles from the inflow edge portion 704 to the outflow edge portion 702. For example, the plurality of first filaments 748 and the plurality of second filaments 749 can each be arranged at a 45 degree angle relative to the inflow edge portion 704. In some examples, the first filaments 748 can be arranged at 90 degree angles relative to the second filaments 749. This arrangement of the first and second filaments 748, 749, as depicted in FIG. 13, can allow the inner skirt 700 to stretch beyond its initial or pre-cut length and diameter when arranged in its annular configuration around an inner surface of a frame of a prosthetic valve.

For example, to deploy a prosthetic valve, such as the prosthetic valve shown in FIG. 16 which includes the inner skirt 700, the frame (e.g., frame 102) is radially expanded to a larger diameter (which can be referred to as the deployment diameter). The deployment diameter can be within a working range of diameters defined between a smallest deployment diameter and a largest deployment diameter. In one non-limiting example, the smallest deployment diameter is about 26 mm and the largest deployment diameter is about 29 mm.

In some examples, when the frame 102 is radially expanded (as shown in FIG. 16, for example), the inner skirt 700 can be slightly stretched in a circumferential direction. In some examples, the inner skirt 700 can be sized such that when the frame 102 is expanded to a diameter within the working range, the inner skirt 700 can be stretched so as to tightly or snugly conform to the inner surface of the frame 102 (such that no slack is formed in the inner skirt 700 around the circumference of the frame 102).

For example, during assembly of the prosthetic valve, the inner skirt 700 can be sutured to the frame 102 when it is expanded to its smallest deployment diameter. The inner skirt 700 can be sized such that it tightly conforms to the inner surface of the frame 102 without any slack in the radial direction when the frame 102 is expanded to its smallest deployment diameter.

When the frame 102 is radially expanded to a larger diameter within the working range of deployment diameters, the inner skirt 700 can further tighten on the frame 102. Thus, in some examples, it can be ensured that the inner skirt 700 does not have any slack in the radial direction when the frame 102 is expanded to any diameter within the working range. In some examples, in order to ensure that the inner skirt 700 fits tightly around the frame 102 at its smallest deployment diameter, the inner skirt 700 can be cut to a smaller size and the inner skirt 700 can be mounted on the frame 102 (at its smallest deployment diameter) with some stretching in the circumferential direction.

In some examples, inner skirt 700 can be sized to fit snugly around the frame without stretching of the skirt in any direction and without any slack in the radial direction when the frame 102 is expanded to its smallest deployment diameter. When the frame is radially expanded to a larger diameter within the working range, the inner skirt 700 can stretch slightly and tighten around the frame.

### Delivery Techniques

For implanting a prosthetic valve within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into and through the descending aorta, around the aortic arch, and through the ascending aorta. The prosthetic valve is positioned within the native aortic valve and radially expanded (e.g., by inflating a balloon, actuating one or more actuators of the delivery apparatus, or deploying the prosthetic valve from a sheath to allow the prosthetic valve to self-expand). Alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Alternatively, in a transaortic procedure, a prosthetic valve (on the distal end portion of the delivery apparatus) is introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. A trial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire previously inserted into a patient's vasculature. Moreover, the disclosed delivery approaches are not intended to be limited. Any of the prosthetic valves disclosed herein can be implanted using any of various delivery procedures and delivery devices known in the art.

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

### Additional Examples of the Disclosed Technology

In view of the above described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A prosthetic heart valve comprising: a radially expandable and compressible frame comprising three axially extending commissure support portions and a plurality of rows of angled struts including a first row of angled struts defining an inflow end of the frame, a second row of angled struts downstream of the first row of angled struts, and a third row of angled struts downstream of the second row of angled struts and defining an outflow end of the frame; wherein downstream ends of the angled struts of the second row of angled struts are connected to each other at a plurality of junctions; wherein the commissure support portions extend from respective junctions of the plurality of junctions of the second row of angled struts to the third row of angled struts; a plurality of leaflets forming three commissures secured to respective commissure support portions of the frame; and an outer skirt disposed around an outer surface of the frame and comprising an inflow edge and an outflow edge, wherein the outflow edge forms three circumferentially spaced projections aligned with the commissure support portions and covering respective junctions of the second row of angled struts, the outflow edge further comprising circumferentially extending sections extending between adjacent projections, wherein the circumferentially extending sections are located upstream of the junctions of the second row of angled struts.

Example 2. The prosthetic heart valve of any example herein, particularly example 1, wherein each projection is shaped to cover the downstream ends of a respective pair of angled struts of the second row of angled struts and a respective junction.

Example 3. The prosthetic heart valve of any example herein, particularly example 2, wherein each projection is shaped as an acute trapezoid that widens from a downstream end of the projection to the circumferentially extending sections.

Example 4. The prosthetic heart valve of any example herein, particularly either example 2 or example 3, wherein each projection is secured to the downstream ends of the respective pair of angled struts of the second row of angled struts and the respective junction with one or more stitches.

Example 5. The prosthetic heart valve of any example herein, particularly any one of examples 1-4, wherein the outer skirt is attached to the frame along a portion of angled struts of the first row of angled struts, the second row of angled struts, and a fourth row of angled struts disposed between the first and second rows of angled struts with sutures, the portion of angled struts extending from one commissure support portion to the inflow end of the frame and from the inflow end to an adjacent commissure support portion.

Example 6. The prosthetic heart valve of any example herein, particularly example 5, wherein cusp edge portions of the plurality of leaflets are attached to the portion of angled struts.

Example 7. The prosthetic heart valve of any example herein, particularly either example 5 or example 6, wherein each projection is attached to a respective commissure support portion by one or more stitches.

Example 8. The prosthetic heart valve of any example herein, particularly any one of examples 1-7, wherein the three projections are pre-formed in the outer skirt.

Example 9. The prosthetic heart valve of any example herein, particularly example 8, wherein the inflow edge of the outer skirt comprises three circumferentially spaced cutouts that are aligned with the projections and that are shaped to follow a shape of angled struts of the first row of angled struts, and wherein each cutout is shaped such that the inflow edge extends toward the outflow edge from straight portions of the inflow edge that are disposed on both sides of the cutout.

Example 10. The prosthetic heart valve of any example herein, particularly example 9, wherein each cutout has a peak at a junction between two angled struts of the first row of angled struts and two angled struts of a fourth row of angled struts disposed downstream of the first row of angled struts, and wherein the outer skirt has a first height defined between the peak of the cutout and a downstream end of a respective projection.

Example 11. The prosthetic heart valve of any example herein, particularly example 10, wherein the outer skirt has a second height defined between the circumferentially extending sections of the outflow edge and the straight portions of the inflow edge, and wherein the first and second heights are the same.

Example 12. The prosthetic heart valve of any example herein, particularly any one of examples 1-7, wherein the outer skirt comprises a stretchable fabric or polymeric material.

Example 13. The prosthetic heart valve of any example herein, particularly example 12, wherein the three projections are formed by stretching the outflow edge of the outer skirt, at regions adjacent to the commissure support portions, over the respective junctions and attaching the stretched projections to the respective commissure support portions with one or more stitches.

Example 14. The prosthetic heart valve of any example herein, particularly example 13, wherein each projection is further attached to downstream end portions of a respective pair of angled struts of the second row of angled struts.

Example 15. The prosthetic heart valve of any example herein, particularly any one of examples 12-14, wherein the inflow edge of the outer skirt extends circumferentially along the inflow end of the frame and is parallel to the circumferentially extending sections of the outflow edge, and wherein a height of the outer skirt, between the inflow edge and the outflow edge, is larger at the projections than between the circumferentially extending sections of the outflow edge and the inflow edge.

Example 16. The prosthetic heart valve of any example herein, particularly any one of examples 1-15, wherein each circumferentially extending section extends in a straight line between two adjacent projections.

Example 17. The prosthetic heart valve of any example herein, particularly any one of examples 1-16, wherein the commissure support portions form commissure windows, wherein leaflets of the plurality of leaflets are connected together at adjacent ends, and wherein each pair of adjacent ends is connected together and secured to a respective commissure window to form a commissure.

Example 18. The prosthetic heart valve of any example herein, particularly example 17, wherein each projection tapers from two adjacent circumferentially extending sections to its apex which is disposed adjacent an upstream edge of a respective commissure.

Example 19. A prosthetic heart valve comprising: a radially expandable and compressible frame comprising three commissure support portions and a plurality of rows of angled struts including a first row of angled struts defining an inflow end of the frame, a second row of angled struts downstream of the first row of angled struts, and a third row of angled struts downstream of the second row of angled struts and defining an outflow end of the frame; wherein downstream ends of the angled struts of the second row of angled struts are connected to each other at a plurality of junctions; a plurality of leaflets forming three commissures secured to respective commissure support portions of the frame, wherein upstream edges of the commissures are located adjacent respective junctions of the second row of struts; and an outer skirt disposed around an outer surface of the frame and comprising an inflow edge and an outflow edge, wherein the outflow edge forms three circumferentially spaced projections aligned with the commissure support portions and covering respective junctions of the second row of angled struts, the outflow edge further comprising straight sections extending between adjacent projections, wherein the straight sections are located upstream of the junctions of the second row of angled struts.

Example 20. The prosthetic heart valve of any example herein, particularly example 19, wherein each projection is shaped to cover the downstream ends of a respective pair of angled struts of the second row of angled struts and a respective junction.

Example 21. The prosthetic heart valve of any example herein, particularly example 20, wherein each projection is A-shaped and widens from a downstream end of the projection to the straight sections, and wherein the downstream end of the projection is disposed adjacent to a respective commissure support portion.

Example 22. The prosthetic heart valve of any example herein, particularly either example 20 or example 21, wherein each projection is secured to the downstream ends of the respective pair of angled struts of the second row of angled struts and the respective junction with one or more stitches.

Example 23. The prosthetic heart valve of any example herein, particularly any one of examples 19-22, wherein the outer skirt is attached to the frame along a portion of angled struts of the first row of angled struts, the second row of angled struts, and a fourth row of angled struts disposed between the first and second rows of angled struts with sutures, the portion of angled struts extending from a first commissure support portion to the inflow end of the frame and from the inflow end to an adjacent, second commissure support portion.

Example 24. The prosthetic heart valve of any example herein, particularly example 23, wherein cusp edge portions of the plurality of leaflets are attached to the portion of angled struts.

Example 25. The prosthetic heart valve of any example herein, particularly any one of examples 19-24, wherein each projection is attached to a respective commissure support portion by one or more stitches.

Example 26. The prosthetic heart valve of any example herein, particularly any one of examples 19-25, wherein the three projections are pre-formed in the outer skirt prior to attaching the outer skirt to the frame.

Example 27. The prosthetic heart valve of any example herein, particularly example 26, wherein the inflow edge of the outer skirt comprises three circumferentially spaced cutouts that are aligned with the projections and that are shaped to follow a shape of angled struts of the first row of angled struts, and wherein each cutout is shaped such that the inflow edge extends toward the outflow edge from straight portions of the inflow edge that are disposed on both sides of the cutout, the straight portions extending along the inflow end of the frame and in parallel to the straight sections of the outflow edge.

Example 28. The prosthetic heart valve of any example herein, particularly example 27, wherein each cutout has a peak at a junction between two angled struts of the first row of angled struts and two angled struts of a fourth row of angled struts disposed downstream of the first row of angled struts, and wherein the outer skirt has a first height defined between the peak of the cutout and a downstream end of a respective projection.

Example 29. The prosthetic heart valve of any example herein, particularly example 28, wherein the outer skirt has a second height defined between the straight sections of the outflow edge and the straight portions of the inflow edge, and wherein the first and second heights are the same.

Example 30. The prosthetic heart valve of any example herein, particularly any one of examples 19-25, wherein the outer skirt comprises a stretchable fabric or polymeric material, and wherein the three projections are formed by stretching the outflow edge of the outer skirt, at regions adjacent to the commissure support portions, over the respective junctions and attaching the stretched projections to the respective commissure support portions with one or more stitches.

Example 31. The prosthetic heart valve of any example herein, particularly example 30, wherein each projection is further attached to downstream end portions of a respective pair of angled struts of the second row of angled struts.

Example 32. The prosthetic heart valve of any example herein, particularly either example 30 or example 31, wherein the inflow edge of the outer skirt extends circumferentially along the inflow end of the frame and is parallel to the straight sections of the outflow edge, and wherein a height of the outer skirt, between the inflow edge and the outflow edge, is larger at the projections than between the straight sections of the outflow edge and the inflow edge.

Example 33. The prosthetic heart valve of any example herein, particularly any one of examples 19-32, wherein the commissure support portions extend axially from respective junctions of the plurality of junctions of the second row of angled struts to the third row of angled struts and form commissure windows, wherein leaflets of the plurality of leaflets are connected together at adjacent ends, and wherein each pair of adjacent ends is connected together and secured to a respective commissure window to form a commissure of the three commissures.

Example 34. The prosthetic heart valve of any example herein, particularly any one of examples 19-32, wherein the commissure support portions are cells of the frame defined by angled struts of the third row of angled struts and a fourth row of angled struts that is disposed between the second and third rows of angled struts.

Example 35. The prosthetic heart valve of any example herein, particularly example 34, wherein each commissure is secured to an attachment member arranged across a respective cell, the attachment member secured to angled struts of the third and fourth rows of angled struts defining the cell.

Example 36. A prosthetic heart valve comprising: a radially expandable and compressible frame comprising three commissure support portions and a plurality of rows of angled struts including a first row of angled struts defining an inflow end of the frame, a second row of angled struts downstream of the first row of angled struts, and a third row of angled struts downstream of the second row of angled struts and defining an outflow end of the frame; wherein downstream ends of the angled struts of the second row of angled struts are connected to each other at a plurality of junctions; a plurality of leaflets forming three commissures secured to respective commissure support portions of the frame, wherein upstream edges of the commissures are located adjacent respective junctions of the second row of angled struts; and an outer skirt disposed around an outer surface of the frame and comprising an inflow edge and an outflow edge, wherein the outflow edge is located upstream of the junctions of the second row of angled struts; and a plurality of commissure extension members located at respective commissure support portions on an outer surface of the frame, wherein each commissure extension member comprises an extension that extends axially upstream of the junctions to cover a gap in the frame that is formed between the respective junction and the outflow edge of the outer skirt.

Example 37. The prosthetic heart valve of any example herein, particularly example 36, wherein an upstream end of the extension is secured to the outflow edge of the outer skirt.

Example 38. The prosthetic heart valve of any example herein, particularly either example 36 or example 37, wherein the extension is secured to angled struts of the second row of angled struts that are connected together by a respective junction.

Example 39. The prosthetic heart valve of any example herein, particularly any one of examples 36-38, wherein the extension overlaps the outflow edge of the outer skirt.

Example 40. The prosthetic heart valve of any example herein, particularly any one of examples 36-39, wherein each commissure extension member comprises a main body forming a respective commissure and secured to a respective commissure support portion, and wherein the extension extends outward, in an upstream direction, from a lower lateral edge of the main body and upstream edge of the respective commissure.

Example 41. The prosthetic heart valve of any example herein, particularly example 40, wherein the main body comprises two laterally extending flaps, and wherein the extension extends outward from a central portion of the main body where the two flaps connect.

Example 42. The prosthetic heart valve of any example herein, particularly example 41, wherein the commissure support portions are axially extending window strut portions that extend axially from respective junctions of the plurality of junctions of the second row of angled struts to the third row of angled struts and form commissure windows therein, and wherein a height of the main body is sized to fit through a respective commissure window.

Example 43. The prosthetic heart valve of any example herein, particularly example 42, wherein the two laterally extending flaps are folded around and secured to respective axially extending window strut portions.

Example 44. The prosthetic heart valve of any example herein, particularly any one of examples 41-43, wherein each commissure is formed by two adjacent ends of two adjacent leaflets of the plurality of leaflets, and wherein each laterally extending flap is coupled to a respective adjacent end of a respective leaflet.

Example 45. The prosthetic heart valve of any example herein, particularly example 44, wherein the commissure extension member is formed as one piece.

Example 46. The prosthetic heart valve of any example herein, particularly example 44, wherein the commissure extension member is formed as two pieces, each piece having one flap of the two flaps, and wherein each piece of the commissure extension member is individually coupled with the respective adjacent end of the respective leaflet.

Example 47. The prosthetic heart valve of any example herein, particularly any one of examples 40-46, wherein the extension tapers from an upstream end of the extension at the outflow edge of the outer skirt to a downstream end of the extension which connects to the main body.

Example 48. The prosthetic heart valve of any example herein, particularly any one of examples 36-47, wherein the extension has a trapezoidal shape that widens toward an upstream end of the extension that connects to the outflow edge of the outer skirt.

Example 49. The prosthetic heart valve of any example herein, particularly any one of examples 36-48, wherein the outer skirt is attached to the frame along a portion of angled struts of the first row of angled struts, the second row of angled struts, and a fourth row of angled struts disposed between the first and second rows of angled struts with sutures, the portion of angled struts extending from one commissure support portion to the inflow end of the frame and from the inflow end to an adjacent commissure support portion.

Example 50. The prosthetic heart valve of any example herein, particularly example 49, wherein cusp edge portions of the plurality of leaflets are attached to the portion of angled struts.

Example 51. The prosthetic heart valve of any example herein, particularly any one of examples 36-50, wherein the outflow edge and the inflow edge of the outer skirt are parallel to each other.

Example 52. A prosthetic heart valve comprising: a frame comprising a plurality of interconnected struts; an inner skirt disposed around an inner surface of the frame and comprising: an inflow edge portion and an outflow edge portion; and opposing first and second edge portions that each extend between the inflow edge portion and the outflow edge portion and are non-perpendicular to the inflow edge portion, wherein the first and second edge portions overlap one another to form an annular configuration of the inner skirt inside the frame; a plurality of leaflets arranged inside the frame, wherein a cusp edge portion of a first leaflet of the plurality of leaflets is arranged along the overlapping first and second edge portions; and a plurality of in-and-out stitches securing the overlapping first and second edge portions and the cusp edge portion of the first leaflet to one another.

Example 53. The prosthetic heart valve of any example herein, particularly example 52, wherein the plurality of interconnected struts of the frame form a plurality of rows of angled struts, and wherein the inner skirt is secured to a portion of angled struts of the plurality of rows of angled struts.

Example 54. The prosthetic heart valve of any example herein, particularly either example 52 or example 53, wherein the first and second edge portions each comprise a single row of spaced apart apertures that extend through a material of the inner skirt, wherein the first and second edge portions overlap one another with their respective apertures overlapping one another, and wherein the plurality of in-and-out stitches extend through the overlapping apertures of the first and second edge portions, and the cusp edge portion of the first leaflet.

Example 55. The prosthetic heart valve of any example herein, particularly any one of examples 52-54, wherein the inner skirt comprises a plurality of spaced apart apertures that undulate along the inner skirt from the first edge portion to the second edge portion, and wherein cusp edge portions of second and third leaflets of the plurality of leaflets are secured to the inner skirt by in-and-out stitches extending through the cusp edge portions of the second and third leaflets and the plurality of spaced apart apertures.

Example 56. The prosthetic heart valve of any example herein, particularly any one of example 52-55, wherein the outflow edge portion of the inner skirt forms three circumferentially spaced projections, and a plurality of circumferentially extending sections extending between adjacent projections.

Example 57. The prosthetic heart valve of any example herein, particularly example 56, wherein the plurality of circumferentially extending sections is a plurality of circumferentially extending folded portions extending between adjacent projections, wherein each folded portion forms a folded edge and a free edge that is arranged against a surface of the inner skirt that faces away from the plurality of leaflets.

Example 58. The prosthetic heart valve of any example herein, particularly either example 56 or example 57, wherein the frame comprises three axially extending commissure support portions, wherein each projection is aligned with and extends to a respective commissure support portion of the three commissure support portions, and wherein the plurality of circumferentially extending sections is located upstream of the three commissure support portions.

Example 59. The prosthetic heart valve of any example herein, particularly any one of examples 52-58, wherein the inner skirt comprises a plurality of first filaments and a plurality of second filaments extending between the inflow edge portion and the outflow edge portion at 45-degree angles.

Example 60. The prosthetic heart valve of any example herein, particularly example 59, wherein the plurality of first filaments is arranged at 90-degree angles relative to the plurality of second filaments.

Example 61. The prosthetic heart valve of any example herein, particularly any one of examples 52-60, further comprising an outer skirt disposed around an outer surface of the frame, the outer skirt having an inflow edge and an outflow edge, wherein the plurality of interconnected struts of the frame form multiple rows of open cells between an outflow end and an inflow end of the frame, and wherein the outflow edge portion of the inner skirt extends through cells of the multiple rows of open cells of the frame to an exterior of the frame and is folded over the outflow edge of the outer skirt.

Example 62. A method for assembling a prosthetic heart valve, comprising: arranging an inner skirt for the prosthetic heart valve into an annular configuration such that opposing first and second edge portions of the inner skirt overlap one another, the first and second edge portions extending between an inflow edge portion and an outflow edge portion of the inner skirt at an angle of less than 90 degrees; arranging a cusp edge portion of a first leaflet of a plurality of leaflets of the prosthetic heart valve along the overlapping first and second edge portions; and securing the overlapping first and second edge portions and the cusp edge portion of the first leaflet to one another with a plurality of in-and-out stitches.

Example 63 The method of any example herein, particularly example 62, wherein arranging the cusp edge portion of the first leaflet along the overlapping first and second end portions includes arranging the cusp edge portion and the first and the second edge portions to be overlapping such that apertures extending through and spaced apart along each of the first and second edge portions and the cusp edge portion align with one another, and wherein securing the overlapping first and second edge portions and the cusp edge portion of the first leaflet to one another includes extending the plurality of in-and-out stitches through the aligned apertures.

Example 64. The method of any example herein, particularly either example 62 or example 63, further comprising securing cusp edge portions of remaining leaflets of the plurality of leaflets to the inner skirt, along a plurality of spaced apart apertures disposed in the inner skirt, the plurality of spaced apart apertures undulating along the inner skirt from the first edge portion to the second edge portion.

Example 65. The method of any example herein, particularly any one of examples 62-64, further comprising securing the inner skirt to an inner surface of an annular frame of the prosthetic heart valve.

Example 66. The method of any example herein, particularly example 65, further comprising folding over the outflow edge portion of the inner skirt and securing a free edge of the outflow edge portion to an outer surface of the inner skirt such that the free edge faces the inner surface of the annular frame.

Example 67. The method of any example herein, particularly example 65, further comprising folding over a plurality of straight sections of the outflow edge portion of the inner skirt to form a plurality of folded portions, extending each folded portion through an open cell of the annular frame that is formed by a plurality of struts of the annular frame, and folding each folded portion over an outflow edge of an outer skirt that is disposed around an outer surface of the frame.

Example 68. The method of any example herein, particularly example 67, wherein the outflow edge portion of the inner skirt forms three circumferentially spaced projections, each projection disposed between two respective, adjacent folded portions of the plurality of folded portions, and further comprising aligning each projection with a respective commissure support portion of three commissure support portions of the annular frame and securing each projection to angled struts that connect to the respective commissure support portion.

Example 69. A prosthetic heart valve comprising: a frame comprising a plurality of interconnected struts forming multiple rows of open cells between an outflow end and an inflow end of the frame; an outer skirt disposed around an outer surface of the frame and comprising an inflow edge and an outflow edge; and an inner skirt disposed around an inner surface of the frame, the inner skirt comprising an inflow edge portion and an outflow edge portion, the outflow edge portion extending through cells of the multiple rows of open cells of the frame to an exterior of the frame and folded over the outflow edge of the outer skirt.

Example 70. The prosthetic heart valve of any example herein, particularly example 69, wherein the outflow edge portion comprises a plurality of folded portions, each folded portion extending through a respective cell of a first row of cells of the multiple rows of open cells of the frame.

Example 71. The prosthetic heart valve of any example herein, particularly example 70, wherein the first row of cells is at least partially defined by struts of the plurality of interconnected struts that define the outflow end of the frame.

Example 72. The prosthetic heart valve of any example herein, particularly either example 70 or example 71, wherein the plurality of interconnected struts includes a first row of angled struts defining the outflow end of the frame and a second row of angled struts disposed upstream of the first row of angled struts, wherein the first and second rows of angled struts at least partially define the first row of cells, and wherein each folded portion folds around two angled struts of the second row of angled struts.

Example 73. The prosthetic heart valve of any example herein, particularly example 72, wherein the plurality of interconnected struts further includes a plurality of axially extending struts spaced circumferentially apart around the frame, and wherein the plurality of axially extending struts extend between the first row of angled struts and the second row of angled struts and form axial sides of the first row of cells.

Example 74. The prosthetic heart valve of any example herein, particularly any one of examples 70-73, wherein each folded portion comprises an inner folded portion that is folded over itself on an inside of the frame, and an outer folded portion that extends from the inner folded portion, through the respective cell and over the outflow edge of the outer skirt.

Example 75. The prosthetic heart valve of any example herein, particularly any one of examples 69-74, wherein the inflow edge portion of the inner skirt is folded around the inflow end of the frame and over the inflow edge of the outer skirt.

Example 76. The prosthetic heart valve of any example herein, particularly any one of examples 69-75, wherein the outflow edge portion of the inner skirt forms three circumferentially spaced projections, and a plurality of circumferentially extending folded portions extending between adjacent projections.

Example 77. The prosthetic heart valve of any example herein, particularly example 76, wherein each folded portion forms a folded edge and a free edge that is arranged over the outflow edge of the outer skirt.

Example 78. The prosthetic heart valve of any example herein, particularly either example 76 or example 77, wherein the frame comprises three axially extending commissure support portions, wherein each projection is aligned with and extends to a respective commissure support portion of the three commissure support portions, and wherein the plurality of circumferentially extending folded portions is located upstream of the three commissure support portions.

Example 79. The prosthetic heart valve of any example herein, particularly example 78, further comprising a plurality of leaflets arranged inside the frame and secured together at adjacent ends to form commissures, each commissure secured to a respective commissure support portion of the three commissure support portions.

Example 80. The prosthetic heart valve of any example herein, particularly example 79, wherein cusp edge portions of the plurality of leaflets are secured to an inner surface of the inner skirt.

Example 81. The prosthetic heart valve of any example herein, particularly any one of examples 69-80, wherein the inner skirt comprises a plurality of first filaments and a plurality of second filaments extending between the inflow edge portion and the outflow edge portion at 45-degree angles.

Example 82. The prosthetic heart valve of any example herein, particularly example 81, wherein the plurality of first filaments is arranged at 90-degree angles relative to the plurality of second filaments.

Example 83. A prosthetic heart valve comprising: a radially expandable and compressible frame comprising a plurality of interconnected struts, the frame having an inflow end and an outflow end; a plurality of leaflets arranged inside the frame; and an inner skirt disposed around an inner surface of the frame and comprising an inflow edge portion and an outflow edge portion, wherein the outflow edge portion is folded over itself such that it forms a folded edge, and a free edge of the outflow edge portion is arranged against a surface of the inner skirt that faces away from the leaflets.

Example 84. The prosthetic heart valve of any example herein, particularly example 83, wherein the folded outflow edge portion comprises a plurality of circumferentially extending folded portions, wherein each folded portion forms a folded edge and a free edge that is arranged against the surface of the inner skirt that faces away from the leaflets.

Example 85. The prosthetic heart valve of any example herein, particularly example 84, wherein the free edge of each folded portion is secured to the surface of the inner skirt by a plurality of stitches.

Example 86. The prosthetic heart valve of any example herein, particularly either example 84 or example 85, wherein the frame comprises three axially extending commissure support portions and a plurality of rows of angled struts including a first row of angled struts defining the inflow end of the frame, a second row of angled struts downstream of the first row of angled struts, and a third row of angled struts downstream of the second row of angled struts and defining the outflow end of the frame, wherein downstream ends of the angled struts of the second row of angled struts are connected to each other at a plurality of junctions, wherein the commissure support portions extend from respective junctions of the plurality of junctions of the second row of angled struts to the third row of angled struts, wherein the plurality of leaflets form three commissures secured to respective commissure support portions of the frame, and wherein the outflow edge portion forms three circumferentially spaced projections aligned with the commissure support portions and extending to respective junctions of the second row of angled struts.

Example 87. The prosthetic heart valve of any example herein, particularly example 86, wherein the plurality of circumferentially extending folded portions extend between adjacent projections of the three circumferentially spaced projection such that each projection is disposed between two respective adjacent folded portions of the plurality of circumferentially extending folded portions.

Example 88. The prosthetic heart valve of any example herein, particularly either example 86 or example 87, wherein the folded edge of each folded portion is located upstream of the junctions of the second row of angled struts.

Example 89. The prosthetic heart valve of any example herein, particularly any one of examples 83-88, wherein the inflow edge portion of the inner skirt wraps around the inflow end of the frame and over an inflow edge of an outer skirt that is disposed around an outer surface of the frame.

Example 90. The prosthetic heart valve of any example herein, particularly any one of examples 83-89, wherein the surface of the inner skirt that the free edge of the outflow edge portion is folded against is disposed against the inner surface of the frame.

Example 91. The prosthetic heart valve of any example herein, particularly any one of examples 83-90, wherein the inner skirt comprises a plurality of first filaments and a plurality of second filaments extending between the inflow edge portion and the outflow edge portion at 45-degree angles, and wherein the plurality of first filaments is arranged at 90-degree angles relative to the plurality of second filaments.

Example 92. A prosthetic heart valve comprising: a frame comprising a plurality of interconnected struts; an inner skirt disposed around an inner surface of the frame and comprising: an inflow edge portion and an outflow edge portion; and opposing first and second edge portions that each extend between the inflow edge portion and the outflow edge portion and are non-perpendicular to the inflow edge portion, wherein the first and second edge portions overlap one another to form an annular configuration of the inner skirt inside the frame; a plurality of leaflets arranged inside the frame, wherein a cusp edge portion of a first leaflet of the plurality of leaflets is arranged along the overlapping first and second edge portions; and a plurality of stitches securing the overlapping first and second edge portions and the cusp edge portion of the first leaflet to one another.

Example 93. A prosthetic heart valve of any one of examples 1-91, wherein the prosthetic heart valve is sterilized.

The features described herein with regard to any example can be combined with other features described in any one or more of the other examples, unless otherwise stated. For example, any one or more of the features of one frame can be combined with any one or more features of another frame. As another example, any one or more features of one outer skirt or attachment member can be combined with any one or more features of another outer skirt or attachment member, respectively.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

We claim:

1. A prosthetic heart valve comprising:
a radially expandable and compressible frame comprising three axially extending commissure support portions and a plurality of rows of angled struts including a first row of angled struts defining an inflow end of the frame, a second row of angled struts downstream of the first row of angled struts, and a third row of angled struts downstream of the second row of angled struts and defining an outflow end of the frame;
wherein downstream ends of the angled struts of the second row of angled struts are connected to each other at a plurality of junctions;
wherein the commissure support portions extend from respective junctions of the plurality of junctions of the second row of angled struts to the third row of angled struts;
a plurality of leaflets forming three commissures secured to respective commissure support portions of the frame; and
an outer skirt disposed around an outer surface of the frame and comprising an inflow edge and an outflow edge, wherein the outflow edge forms three circumferentially spaced projections aligned with the commissure support portions and covering respective junctions of the second row of angled struts, the outflow edge further comprising circumferentially extending sections extending between adjacent projections, wherein the circumferentially extending sections are located upstream of the junctions of the second row of angled struts.

2. The prosthetic heart valve of claim 1, wherein each projection is shaped to cover the downstream ends of a respective pair of angled struts of the second row of angled struts and a respective junction.

3. The prosthetic heart valve of claim 2, wherein each projection is shaped as an acute trapezoid that widens from a downstream end of the projection to the circumferentially extending sections.

4. The prosthetic heart valve of claim 2, wherein each projection is secured to the downstream ends of the respective pair of angled struts of the second row of angled struts and the respective junction with one or more stitches.

5. The prosthetic heart valve of claim 1, wherein the outer skirt is attached to the frame along a portion of angled struts of the first row of angled struts, the second row of angled struts, and a fourth row of angled struts disposed between the first and second rows of angled struts with sutures, the portion of angled struts extending from one commissure support portion to the inflow end of the frame and from the inflow end to an adjacent commissure support portion.

6. The prosthetic heart valve of claim 5, wherein each projection is attached to a respective commissure support portion by one or more stitches.

7. The prosthetic heart valve of claim 1, wherein the inflow edge of the outer skirt comprises three circumferentially spaced cutouts that are aligned with the projections and that are shaped to follow a shape of angled struts of the first row of angled struts, and wherein each cutout is shaped such that the inflow edge extends toward the outflow edge from straight portions of the inflow edge that are disposed on both sides of the cutout.

8. The prosthetic heart valve of claim 1, wherein the outer skirt comprises a stretchable fabric or polymeric material.

9. The prosthetic heart valve of claim 8, wherein the three projections are formed by stretching the outflow edge of the outer skirt, at regions adjacent to the commissure support portions, over the respective junctions and attaching the stretched projections to the respective commissure support portions with one or more stitches.

10. A prosthetic heart valve comprising:
a radially expandable and compressible frame comprising three commissure support portions and a plurality of rows of angled struts including a first row of angled struts defining an inflow end of the frame, a second row of angled struts downstream of the first row of angled struts, and a third row of angled struts downstream of the second row of angled struts and defining an outflow end of the frame;
wherein downstream ends of the angled struts of the second row of angled struts are connected to each other at a plurality of junctions;
a plurality of leaflets forming three commissures secured to respective commissure support portions of the frame, wherein upstream edges of the commissures are located adjacent respective junctions of the second row of struts; and
an outer skirt disposed around an outer surface of the frame and comprising an inflow edge and an outflow edge, wherein the outflow edge forms three circumferentially spaced projections aligned with the commissure support portions and covering respective junctions of the second row of angled struts, the outflow edge further comprising straight sections extending between adjacent projections, wherein the straight sections are located upstream of the junctions of the second row of angled struts.

11. The prosthetic heart valve of claim 10, wherein each projection is shaped to cover the downstream ends of a respective pair of angled struts of the second row of angled struts and a respective junction.

12. The prosthetic heart valve of claim 11, wherein each projection is A-shaped and widens from a downstream end of the projection to the straight sections, and wherein the downstream end of the projection is disposed adjacent to a respective commissure support portion.

13. The prosthetic heart valve of claim 10, wherein the outer skirt is attached to the frame along a portion of angled struts of the first row of angled struts, the second row of angled struts, and a fourth row of angled struts disposed between the first and second rows of angled struts with sutures, the portion of angled struts extending from a first commissure support portion to the inflow end of the frame and from the inflow end to an adjacent, second commissure support portion.

14. A prosthetic heart valve comprising:
a frame comprising a plurality of interconnected struts;
an inner skirt disposed around an inner surface of the frame and comprising:
an inflow edge portion and an outflow edge portion; and
opposing first and second edge portions that each extend between the inflow edge portion and the outflow edge portion and are non-perpendicular to the inflow edge portion, wherein the first and second edge portions overlap one another to form an annular configuration of the inner skirt inside the frame;
a plurality of leaflets arranged inside the frame, wherein a cusp edge portion of a first leaflet of the plurality of leaflets is arranged along the overlapping first and second edge portions; and
a plurality of stitches securing the overlapping first and second edge portions and the cusp edge portion of the first leaflet to one another.

15. The prosthetic heart valve of claim 14, wherein the plurality of interconnected struts of the frame form a plurality of rows of angled struts, and wherein the inner skirt is secured to a portion of angled struts of the plurality of rows of angled struts.

16. The prosthetic heart valve of claim 14, wherein the first and second edge portions each comprise a single row of spaced apart apertures that extend through a material of the inner skirt, wherein the first and second edge portions overlap one another with their respective apertures overlapping one another, and wherein the plurality of stitches are in-and-out stitches that extend through the overlapping apertures of the first and second edge portions, and the cusp edge portion of the first leaflet.

17. The prosthetic heart valve of claim 14, wherein the inner skirt comprises a plurality of spaced apart apertures that undulate along the inner skirt from the first edge portion to the second edge portion, and wherein cusp edge portions of second and third leaflets of the plurality of leaflets are secured to the inner skirt by in-and-out stitches extending through the cusp edge portions of the second and third leaflets and the plurality of spaced apart apertures.

18. The prosthetic heart valve of claim 14, wherein the outflow edge portion of the inner skirt forms three circumferentially spaced projections, and a plurality of circumferentially extending sections extending between adjacent projections.

19. The prosthetic heart valve of claim 18, wherein the plurality of circumferentially extending sections is a plurality of circumferentially extending folded portions extending between adjacent projections, wherein each folded portion forms a folded edge and a free edge that is arranged against a surface of the inner skirt that faces away from the plurality of leaflets.

20. The prosthetic heart valve of claim 18, wherein the frame comprises three axially extending commissure support portions, wherein each projection is aligned with and extends to a respective commissure support portion of the three commissure support portions, and wherein the plurality of circumferentially extending sections is located upstream of the three commissure support portions.
